(19) United States
(12) Patent Application Publication
(10) Pub. No.: US 2009/0181939 A1
(43) Pub. Date: Jul. 16, 2009

(54) Azetidinecarboxamide Derivatives And Their Use In The Treatment Of CB1 Receptor Mediated Disorders

(75) Inventors: James Edward Paul Davidson, Winnersh (GB); Jonathan Mark Bentley, Winnersh (GB); Claire Elizabeth Dawson, Winnersh (GB); Kerry Harrison, Winnersh (GB); Howard Langham Mansell, Winnersh (GB); Alan Leslie Pither, Winnersh (GB); Robert Mark Pratt, Winnersh (GB); Jonathan Richard Anthony Roffey, Winnersh (GB); Victoria Jane Ruston, Winnersh (GB)

Correspondence Address: BANNER & WITCOFF, LTD., 1100 13th STREET, N.W., SUITE 1200, WASHINGTON, DC, 20005-4051, US

(73) Assignee: VERNALIS RESEARCH LIMITED., Winnersh (GB)

(21) Appl. No.: 12/407,021

(22) Filed: Mar. 19, 2009

Related U.S. Application Data
(63) Continuation of application No. 10/554,447, filed on Oct. 12, 2006, now abandoned.

(30) Foreign Application Priority Data
May 1, 2003 (GB) 0310052.6

(51) Int. Cl. A61K 31/551; C07D 403/02; A61K 31/397; C07D 205/04

(52) U.S. Cl. 514/210.18; 540/575; 514/210.17; 548/953

(57) ABSTRACT

Compounds of formula (I) and their use in therapy, particularly for the treatment of a disorder mediated by CB₁ receptors, such as obesity, wherein: R¹ is aryl or heteroaryl; R² is alkyl, aryl or heteroaryl; R³ is alkyl, aryl, heteroaryl, NR⁹R¹⁰, OR¹⁵, or NR¹⁶C(O)R¹⁷; Y is C═O, C═S, SO₂, or (CR⁷R⁸)ₚ; m=1 or 2; n=1 or 2; and p=1, 2, 3 or 4, R⁷ to R¹⁷ being as defined in the specification; wherein if —Y—R³ is C(O)NH(alkyl) then: R¹ and/or R² is selected from heteroaryl; and/or m and/or n is 2; and/or R¹¹ and/or R¹² is lower alkyl, or a pharmaceutically acceptable salt or prodrug thereof.

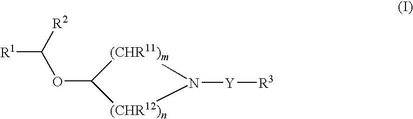

The present invention relates primarily to new chemical compounds for the treatment of disorders mediated by the cannabinoid CB₁ receptor, particularly to the treatment of obesity and other eating disorders associated with excessive food intake.

It has been recognised that obesity is a disease process influenced by environmental factors in which the traditional weight loss methods of dieting and exercise need to be supplemented by therapeutic products (S. Parker, “Obesity: Trends and Treatments”, Scrip Reports, PJB Publications Ltd, 1996).

Whether someone is classified as overweight or obese is generally determined on the basis of their body mass index (BMI) which is calculated by dividing body weight (kg) by height squared (m²). Thus, the units of BMI are kg/m² and it is possible to calculate the BMI range associated with minimum mortality in each decade of life. Overweight is defined as a BMI in the range 25-30 kg/m², and obesity as a BMI greater than 30 kg/m². There are problems with this definition in that it does not take into account the proportion of body mass that is muscle in relation to fat (adipose tissue). To account for this, obesity can also be defined on the basis of body fat content: greater than 25% and 30% in males and females, respectively.

As the BMI increases there is an increased risk of death from a variety of causes that is independent of other risk factors. The most common diseases with obesity are cardiovascular disease (particularly hypertension), diabetes (obesity aggravates the development of diabetes), gall bladder disease (particularly cancer) and diseases of reproduction. Research has shown that even a modest reduction in body weight can correspond to a significant reduction in the risk of developing coronary heart disease.

Compounds marketed as anti-obesity agents include Orlistat (Reductil®) and Sibutramine. Orlistat (a lipase inhibitor) inhibits fat absorption directly and tends to produce a high incidence of unpleasant (though relatively harmless) side-effects such as diarrhoea. Sibutramine (a mixed 5-HT/noradrenaline reuptake inhibitor) can increase blood pressure and heart rate in some patients. The serotonin releaser/reuptake inhibitors fenfluramine (Pondimin®) and dexfenfluramine (Redux™) have been reported to decrease food intake and body weight over a prolonged period (greater than 6 months). However, both products were withdrawn after reports of preliminary evidence of heart valve abnormalities associated with their use. There is therefore a need for the development of a safer anti-obesity agent.

There now exists extensive pre-clinical and clinical data supporting the use of CB₁ receptor antagonists/inverse agonists for the treatment of obesity.

Preparations of marijuana (Cannabis sativa) have been used for over 5000 years for both medicinal and recreational purposes. The major psychoactive ingredient of marijuana has been identified as □⁹-tetrahydrocannabinol (□⁹-THC), one of a member of over 60 related cannabinoid compounds isolated from this plant. It has been demonstrated that □⁹-THC exerts its effects via agonist interaction with cannabinoid (CB) receptors. So far, two cannabinoid receptor subtypes have been characterised (CB₁ and CB₂). The CB₁ receptor subtype is found predominantly in the central nervous system, and to a lesser extent in the peripheral nervous system and various peripheral organs. The CB₂ receptor subtype is found predominantly in lymphoid tissues and cells. To date, three endogenous agonists (endocannabinoids) have been identified which interact with both CB₁ and CB₂ receptors (anandamide, 2-arachidonyl glycerol and noladin ether).

Genetically obese rats and mice exhibit markedly elevated endocannabinoid levels in brain regions associated with ingestive behaviour (Di Marzo et al. 2001 Nature 410: 822-825). Furthermore, increased levels of endocannabinoids are observed upon the fasting of normal, lean animals (Kirkham et al., British Journal of Pharmacology 2002, 136(4) 550-557). Exogenous application of endocannabinoids leads to the same physiological effects observed with □⁹-THC treatment, including appetite stimulation (Jamshida et al., British Journal of Pharmacology 2001, 134: 1151-1154), analgesia, hypolocomotion, hypothermia, and catalepsy.

CB₁ (CB₁^{-/-}) and CB₂ (CB₂^{-/-}) receptor knockout mice have been used to elucidate the specific role of the two cannabinoid receptor subtypes. Furthermore, for ligands such as □⁹-THC which act as agonists at both receptors, these mice have allowed identification of which receptor subtype is mediating specific physiological effects. CB₁^{-/-}, but not CB₂^{-/-}, mice are resistant to the behavioural effects of agonists such as □⁹-THC. CB₁^{-/-} animals have also been shown to be resistant to both the body weight gain associated with chronic high fat diet exposure, and the appetite-stimulating effects of acute food deprivation.

These findings suggest a clear role for both endogenous and exogenous cannabinoid receptor agonists in increasing food intake and body weight via selective activation of the CB₁ receptor subtype.

The therapeutic potential for cannabinoid receptor ligands has been extensively reviewed (Exp. Opin. Ther. Pat. 1998, 8, 301-313; Exp. Opin. Ther. Pat. 2000, 10, 1529-1538; Trends in Pharm. Sci. 2000, 21, 218-224; Exp. Opin. Ther. Pat. 2002, 12(10), 1475-1489).

At least one compound (SR-141716A) characterised as a CB₁ receptor antagonist/inverse agonist is known to be in clinical trials for the treatment of obesity.

WO-00/15609, WO-01/64632, WO-01/64633 and WO-01/64634 disclose azetidine derivatives as CB₁ receptor antagonists. WO 02/28346 discloses the association of an azetidine derivative as a CB₁ receptor antagonist, and sibutramine, for the treatment of obesity. Azetidine carboxamides have also been proposed for use in the treatment of anxiety and epilepsy (WO-99/37612) and for neuroprotection (WO-01/07023).

There remains a medical need for low molecular weight CB₁ receptor antagonists/inverse agonists with pharmacokinetic and pharmacodynamic properties making them suitable for use as pharmaceutical agents. There also remains a medical need for new treatments of disorders mediated by the CB₁ receptor, particularly eating disorders, and particularly obesity. The object of the present invention is to provide such pharmaceutical agents and treatments.

New compounds have now been found which show unexpected efficacy as anti-obesity agents. These compounds have been shown to selectively bind to the CB₁ receptor subtype with high affinity. Such compounds have been shown to dose-dependently block the effects of an exogenously applied cannabinoid receptor agonist (eg ?⁹-THC) in mice.

Furthermore, such compounds have been shown to reduce food intake and body weight gain in both rat and mouse models of feeding behaviour.

According to the present invention, there is provided the use of a compound of formula (I) or a pharmaceutically acceptable salt or prodrug thereof in the manufacture of a medicament for the treatment of disorders mediated by CB1 receptors:

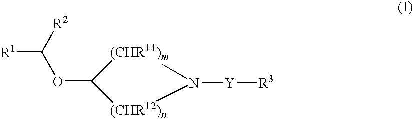

wherein:
R¹ is aryl or heteroaryl;
R² is alkyl, aryl or heteroaryl;
R³ is alkyl, aryl, heteroaryl, NR⁹R¹⁰, OR¹⁵, or NR¹⁶C(O)R¹⁷;
Y is C═O, C═S, SO₂, or (CR⁷R⁸)ₚ;
R⁷ and R⁸ are independently selected from H and lower alkyl;
R⁹ is selected from alkyl, aryl, heteroaryl, and non-aromatic heterocyclic groups, or together with R¹⁰ forms a saturated 4, 5, 6 or 7 membered ring optionally containing an additional heteroatom selected from N and O;
R¹⁰ is selected from H and lower alkyl, or together with R⁹ forms a saturated 4, 5, 6 or 7 membered ring optionally containing an additional heteroatom selected from N and O;
R¹¹ and R¹² are independently selected from H and lower alkyl;
R¹⁵ is selected from alkyl and aryl;
R¹⁶ is selected from H and lower alkyl;
R¹⁷ is selected from alkyl, aryl and heteroaryl;
m=1 or 2;
n=1 or 2; and
p=1, 2, 3 or 4,
wherein if —Y—R³ is —C(O)NH(alkyl) then:
R¹ and/or R² is selected from heteroaryl; and/or
m and/or n is 2; and/or
R¹¹ and/or R¹² is lower alkyl.

According to a second aspect of the invention there is provided a compound of formula (I).

The active compounds with which the invention is concerned are antagonists and/or inverse agonists at the cannabinoid-1 (CB₁) receptor and are useful for the treatment, prevention and suppression of diseases mediated by the CB₁ receptor. The invention is concerned with the use of these compounds to selectively antagonise the CB₁ receptor and, as such, in the treatment of obesity and other disorders.

Reference in the present specification to an “alkyl” group means a branched or unbranched, cyclic or acyclic, saturated or unsaturated (e.g. alkenyl (including allyl) or alkynyl (including propargyl)) hydrocarbyl radical. Where cyclic or acyclic the alkyl group is preferably C₁ to C₁₂, more preferably C₁ to C₈ (such as methyl, ethyl, propyl, isopropyl, butyl, isobutyl, tert-butyl, sec-butyl, pentyl, isopentyl, hexyl, heptyl, octyl). It will be appreciated therefore that the term “alkyl” as used herein includes alkyl (branched or unbranched), alkenyl (branched or unbranched), alkynyl (branched or unbranched), cycloalkyl, cycloalkenyl and cycloalkynyl. A cyclic alkyl group may be a mono-bridged or multiply-bridged cyclic alkyl group. In a preferred embodiment, a cyclic alkyl group is preferably C₃ to C₁₂, more preferably C₅ to C₈ and an acyclic alkyl group is preferably C₁ to C₁₀, more preferably C₁ to C₆, more preferably methyl, ethyl, propyl (n-propyl or isopropyl), butyl (n-butyl, isobutyl, tertiary butyl or sec-butyl) or pentyl (including n-pentyl and iso-pentyl), more preferably methyl.

As used herein, the term “lower alkyl” means a branched or unbranched, cyclic or acyclic, saturated or unsaturated (e.g. alkenyl or alkynyl)hydrocarbyl radical wherein said cyclic lower alkyl group is C₅, C₆ or C₇, and wherein said acyclic lower alkyl group is C₁, C₂, C₃ or C₄. It will be appreciated therefore that the term “lower alkyl” as used herein includes lower alkyl (branched or unbranched), lower alkenyl (branched or unbranched), lower alkynyl (branched or unbranched), cycloloweralkyl, cycloloweralkenyl and cycloloweralkynyl. Preferably, a lower alkyl group is preferably selected from methyl, ethyl, propyl (n-propyl or isopropyl) or butyl (n-butyl, isobutyl, tertiary-butyl or sec-butyl), more preferably methyl.

Reference in the present specification to an “aryl” group means a mono or bicyclic aromatic group, such as phenyl or naphthyl, and preferably a mono-cyclic aromatic group.

Reference in the present specification to a “heteroaryl” group means an aromatic group containing one or more heteroatoms, preferably 1, 2 or 3 heteroatoms, preferably 1 or 2 heteroatoms. Preferably the heteroatoms are selected from O, S and N, preferably from O and N. Preferably the heteroaryl group comprises 5 or 6-membered ring systems. The heteroaryl group is preferably a monocyclic or bicyclic ring system, preferably monocyclic. Examples include thienyl, furanyl, pyrrolyl, imidazolyl, pyrazolyl, thiazolyl, isothiazolyl, oxazolyl, isooxazolyl, pyridyl, pyrazinyl, pyrimidinyl, quinolinyl, isoquinolinyl, benzofuranyl and isobenzofuryl.

Reference in the present specification to a non-aromatic heterocylic group is to a saturated or partially unsaturated 4, 5, 6 or 7-membered ring containing 1, 2 or 3 heteroatoms selected from N, O and S, preferably 1 or 2 heteroatoms, preferably selected from N and O. Examples include piperidinyl, morpholinyl, piperazinyl and pyrrolidinyl.

The alkyl, aryl, heteroaryl and non-aromatic heterocyclic groups may be substituted or unsubstituted. In one embodiment, only the alkyl, aryl, heteroaryl and non-aromatic heterocyclic groups defined above as R¹ to R²⁰ may be substituted. Where R⁹ and R¹⁰ together form a 4, 5, 6 or 7-membered ring, the ring may be substituted or unsubstituted. Where R¹³ and R¹⁴ together form a 5 or 6-membered ring, the ring may be substituted or unsubstituted. Where substituted, there will generally be 1 to 3 substituents present, preferably 1 or 2 substituents. Substituents may include:

carbon containing groups such as
alkyl
aryl, arylalkyl (e.g. substituted and unsubstituted phenyl, substituted and unsubstituted benzyl);
   halogen atoms and halogen containing groups such as
haloalkyl (e.g. trifluoromethyl);
   oxygen containing groups such as
alcohols (e.g. hydroxy, hydroxyalkyl, (aryl)(hydroxy)alkyl),
ethers (e.g. alkoxy, alkoxyalkyl, aryloxyalkyl),
aldehydes (e.g. carboxaldehyde),
ketones (e.g. alkylcarbonyl, alkylcarbonylalkyl, arylcarbonyl, arylalkylcarbonyl, arylcarbonylalkyl),
acids (e.g. carboxy, carboxyalkyl),
acid derivatives such as esters
   (e.g. alkoxycarbonyl, alkoxycarbonylalkyl, alkylcarbonyloxy, alkylcarbonyloxyalkyl) and amides
   (e.g. aminocarbonyl, mono- or dialkylaminocarbonyl, aminocarbonylalkyl, mono- or dialkylaminocarbonylalkyl, arylaminocarbonyl);
      nitrogen containing groups such as
amines (e.g. amino, mono- or dialkylamino, aminoalkyl, mono- or dialkylaminoalkyl),
azides,
nitriles (e.g. cyano, cyanoalkyl),
nitro;
   sulphur containing groups such as
thiols, thioethers, sulphoxides and sulphones
   (e.g. alkylthio, alkylsulfinyl, alkylsulfonyl, alkylthioalkyl, alkylsulfinylalkyl, alkylsulfonylalkyl, arylthio, arylsulfinyl, arylsulfonyl, arylthioalkyl, arylsulfinylalkyl, arylsulfonylalkyl);
      and heterocyclic groups containing one or more, preferably one, heteroatom,
(e.g. thienyl, furanyl, pyrrolyl, imidazolyl, pyrazolyl, thiazolyl, isothiazolyl, oxazolyl, pyrrolidinyl, pyrrolinyl, imidazolidinyl, imidazolinyl, pyrazolidinyl, tetrahydrofuranyl, pyranyl, pyronyl, pyridyl, pyrazinyl, pyridazinyl, piperidyl, piperazinyl, morpholinyl, thionaphthyl, benzofuranyl, isobenzofuryl, indolyl, oxyindolyl, isoindolyl, indazolyl, indolinyl, 7-azaindolyl, isoindazolyl, benzopyranyl, coumarinyl, isocoumarinyl, quinolyl, isoquinolyl, naphthridinyl, cinnolinyl, quinazolinyl, pyridopyridyl, benzoxazinyl, quinoxadinyl, chromenyl, chromanyl, isochromanyl and carbolinyl).

Where an aryl group is phenyl, the phenyl may be substituted by adjacent substituents forming a 5 or 6 membered saturated ring optionally containing 1 or 2 heteroatoms, preferably selected from N, O and S, preferably from N and O. Where the saturated ring contains 2 nitrogen atoms, the ring is preferably a 6-membered ring. Where the saturated ring contains 2 oxygen atoms, the ring may be a 5- or 6-membered ring. Examples include 2,3-dihydrobenzo[b]furan-7-yl, 2,3-dihydrobenzo[b]thiophen-6-yl, 1,2,3,4-tetrahydronaphthalen-5-yl, 2,3-dihydrobenzo[1,4]dioxin-6-yl and 1,2,3,4-tetrahydroisoquinolin-8-yl.

Preferred substituents include allyl (including haloalkyl), alkoxy (including haloalkoxy), aryl, nitrile or halo. Preferred halogen-containing groups include trifluoromethyl.

As used herein, the term “alkoxy” means alkyl-O— and “alkoyl” means alkyl-CO—.

As used herein, the term “halogen” means a fluorine, chlorine, bromine or iodine radical, preferably a fluorine or chlorine radical.

As used herein, the term “prodrug” means any pharmaceutically acceptable prodrug of the compound of formula (I). For example, the compound of formula (I) may be prepared in a prodrug form wherein a free —OH group is derivatised (for example, via an ester, amide or phosphate bond) with a suitable group (the group may contain, for example, an alkyl, aryl, phosphate, sugar, amine, glycol, sulfonate or acid function) which is suitably labile so as it will be removed/cleaved (eg. by hydrolysis) to reveal the compound of formula (I) sometime after administration or when exposed to the desired biological environment.

As used herein, the term “pharmaceutically acceptable salt” means any pharmaceutically acceptable salt of the compound of formula (I). Salts may be prepared from pharmaceutically acceptable non-toxic acids and bases including inorganic and organic acids and bases. Such acids include acetic, benzenesulfonic, benzoic, camphorsulfonic, citric, ethenesulfonic, dichloroacetic, furnaric, gluconic, glutamic, hippuric, hydrobromic, hydrochloric, isethionic, lactic, maleic, malic, mandelic, methanesulfonic, mucic, nitric, pamoic, pantothenic, phosphoric, succinic, sulfuric, tartaric, oxalic, p-toluenesulfonic and the like. Particularly preferred are hydrochloric, hydrobromic, phosphoric, sulfuric and methanesulfonic acids, and most particularly preferred is the methanesulfonate salt. Acceptable base salts include alkali metal (e.g. sodium, potassium), alkaline earth metal (e.g. calcium, magnesium) and aluminium salts.

The compounds with which the invention is concerned may exist in a number of diastereomeric and/or enantiomeric forms. Unless otherwise stated, reference in the present specification to “a compound of formula (I)” is a reference to all stereoisomeric forms of the compound and includes a reference to the unseparated stereoisomers in a mixture, racemic or non-racemic, and to each stereoisomer in its pure form.

In the compounds with which the invention is concerned, preferably R¹ and/or R² is substituted, preferably with 1 to 3 substituents and most preferably with 1 or 2 substituents.

In one embodiment of the compounds with which the invention is concerned, R¹ and R² are independently selected from a group -A(R⁴)(R⁵)(R⁶), where A is an aryl or heteroaryl ring, and where A may be selected from phenyl, naphthyl, thienyl, furanyl, pyrrolyl, imidazolyl, pyrazolyl, thiazolyl, isothiazolyl, oxazolyl, isooxazolyl, pyridyl, pyrazinyl, pyrimidinyl, quinolinyl, isoquinolinyl, benzofuranyl and isobenzofuryl. Preferably, one of R¹ and R² is aryl and the other is heteroaryl, or both R¹ and R² are aryl. Preferably both R¹ and R² are monocyclic. In this embodiment, R⁴, R⁵ and R⁶ are independently selected from hydrogen, halo, alkyl (including haloalkyl), thioalkyl, alkoxy (including haloalkoxy), alkylsulfonyl, amino, mono- and di-alkyl amino, mono- and di-aryl amino, alkylarylamino, aminoalkyl, alkylaminoalkyl, dialkylaminoalkyl, NR¹⁸C(O)R¹⁹, NR¹⁸SO₂R²⁰, COOR¹⁹, OC(O)R²⁰, CONR¹³R¹⁴ and SO₂NR¹³R¹⁴, wherein R¹³ and R¹⁴ are independently selected from hydrogen and alkyl or may form a 5 or 6 membered ring optionally containing 1 or 2 additional heteroatoms selected from N, O and S; and wherein R¹⁸ is selected from H and lower alkyl (preferably H), R¹⁹ is selected from H, alkyl, aryl and heteroaryl (preferably alkyl, preferably lower alkyl) and R²⁰ is selected from alkyl, aryl and heteroaryl (preferably alkyl, preferably lower alkyl). The groups R¹ and R² may be the same or different, and in one embodiment are different.

Where R⁴, R⁵ and R⁶ are selected from halo, the halo group is preferably fluoro, chloro, bromo or iodo, preferably chloro or bromo. Where R⁴, R⁵ and R⁶ are selected from alkyl, thioalkyl, alkoxy and alkylsulfonyl, the alkyl is preferably selected from lower alkyl, and preferably from methyl and ethyl, preferably methyl. Where R⁴, R⁵ and R⁵ are selected from haloakyl, the alkyl is preferably methyl, and the R⁴, R⁵ or R⁶ group is preferably trifluoromethyl. Where R⁴, R⁵ and R⁶ are selected from haloalkoxy, the alkyl is preferably methyl and the R⁴, R⁵ or R⁶ group is preferably selected from trifluoromethoxy or difluoromethoxy, preferably difluoromethoxy. Preferably one or two of R⁴, R⁵ and R⁶ are hydrogen. Preferably, at least one of the R¹ and R² groups has a non-hydrogen substituent in the ortho-position(s) relative to the point of attachment to the [—CH—O—] group. The R¹ or R² groups may independently have one or two non-hydrogen substituents in said ortho position(s). Preferred ortho-substituents include halo and haloalkyl, as described herein. Particularly preferred ortho-substituents are chloro and trifluoromethyl, particularly trifluoromethyl.

Where R¹³ and R¹⁴ form a 5- or 6-membered ring, the ring is preferably 6-membered, and is preferably saturated or partially saturated, preferably saturated. Where the ring contains additional heteroatoms, these are preferably selected from N and O. Preferably there are 0 or 1 additional heteroatoms.

In the compounds with which the invention is concerned, preferably R¹ is selected from aryl.

In the compounds with which the invention is concerned, preferably R² is selected from aryl or heteroaryl.

In the compounds with which the invention is concerned, preferably R³ is selected from NR⁹R¹⁰. In an alternative embodiment R³ is selected from alkyl, aryl and heteroaryl, particularly wherein Y is selected from SO₂ and (CR⁷R⁸)ₚ.

In the compounds with which the invention is concerned, preferably Y is selected from C═O, C═S and SO₂, preferably C═O and SO₂, preferably C═O. Where Y is selected from (CR⁷R⁸)ₚ, then R⁷ and/or R⁸ are preferably hydrogen or methyl, preferably hydrogen, and p is preferably 1 or 2, preferably 1.

Where Y is SO₂, R³ is preferably selected from alkyl, aryl and heteroaryl.

Where Y is (CR⁷R⁸)ₚ, particularly wherein p is 1, R³ is preferably selected from allyl, aryl, heteroaryl.

In the compounds with which the invention is concerned, where R⁹ is a non-aromatic heterocyclic group, it is preferably selected from piperidinyl (preferably 1-piperidinyl) and morpholinyl (preferably 4-morpholinyl).

Where R⁹ is cyclic, as defined herein, particularly wherein R⁹ is aryl or heteroaryl, particularly aryl, and particularly phenyl, the R⁹ group may be substituted with one or more substituent groups, preferably one substituent group, and particularly with halo (preferably chloro and fluoro), nitro, alkoxy (preferably alkoxy) and haloalkyl (preferably trifluoromethyl), and particularly halo.

In one embodiment, the ring formed by NR⁹R¹⁰ may be substituted, and preferred substituents include hydroxy, methoxy, mono- and di-alkyl amino and alkoxycarbonyl.

In one embodiment (hereinafter referred to as embodiment (i)), R⁹ is selected from aryl, heteroaryl and a non-aromatic heterocyclic group, and R¹⁰ is selected from H and lower alkyl. In an alternative embodiment (hereinafter referred to as embodiment (ii)), R⁹ is selected from alkyl and R¹⁰ is selected from lower alkyl. In a further alternative embodiment (hereinafter referred to as embodiment (iii)), R⁹ and R¹⁰ form a 4, 5, 6 or 7-membered ring, preferably a 5, 6 or 7-membered ring, optionally containing an additional heteroatom selected from N and O, but preferably not containing such an additional heteroatom.

In the compounds with which the invention is concerned, preferably m is 1 and/or n is 1. Preferably both m and n are 1. Where m is 2, the R¹¹ groups may be the same or different, but at least one of the R¹¹ groups in the (CHR¹¹)₂ moiety is hydrogen. Where n is 2, the R¹² groups may be the same or different, but at least one and preferably both of the R¹² groups in the (CHR¹²)₂ moiety is/are hydrogen.

In the compounds with which the invention is concerned, preferably R¹¹ and R¹² are independently selected from hydrogen and methyl. Preferably, at least one of R¹¹ and R¹² is hydrogen.

Where R¹⁵ is selected from alkyl, it is preferably lower alkyl (substituted or unsubstituted). Where R¹⁵ is selected from aryl, it is preferably phenyl (substituted or unsubstituted). In one embodiment, R¹⁵ is selected from lower alkyl, benzyl and phenyl, preferably lower alkyl and benzyl and preferably lower alkyl.

Preferably R¹⁶ is hydrogen.

Preferably R¹⁷ is lower alkyl, aryl or heteroaryl, and in one embodiment is aryl, particularly phenyl.

Of these, the preferred compounds are:
4-[3-(2,4′-dichlorobenzhydryloxy)azetidine-1-carbonyl]-[1,4]diazepane-1-carboxylic acid tert-butyl ester
3-(2,4′-dichlorobenzhydryloxy)-N-methyl-N-pentyl-azetidine-1-carboxamide
3-(2,4,4′-trichlorobenzhydryloxy)-N-methyl-N-benzyl-azetidine-1-carboxamide
3-[2-(trifluoromethyl)-4′-chlorobenzhydryloxy]-N-methyl-N-benzyl-azetidine-1-carboxamide
3-(2-methyl-4′-chlorobenzhydryloxy)-N-methyl-N-benzyl-azetidine-1-carboxamide
3-(2,4′-dichlorobenzhydryloxy)-N-(tert-butyl)-azetidine-1-thiocarboxamide
3-[(S*)-2,4′-dichlorobenzhydryloxy]-N-(tert-butyl)-azetidine-1-thiocarboxamide
3-[2-(trifluoromethyl)-4′-chlorobenzhydryloxy]-N-(tert-butyl)-azetidine-1-thiocarboxamide
3-(2,4,4′-trichlorobenzhydryloxy)-1-[(S)-2-methoxy-2-phenylethanoyl]azetidine
3-(2,4′-dichlorobenzhydryloxy)-1-(3-chlorothiophen-2-yl-formyl)azetidine
1-[2-(tert-butyl)acetyl]-3-[2-(trifluoromethyl)-a-(2,3-dihydrobenzo[1,4]dioxin-6-yl)benzyloxy]azetidine
1-(4-nitrophenylsulfonyl)-3-[2-(trifluoromethyl)-4′-chlorobenzhydryloxy]azetidine
1-(thiophen-3-ylsulfonyl)-3-[2-(trifluoromethyl)-4′-chlorobenzhydryloxy]azetidine
1-(3-fluorophenylsulfonyl)-3-[2-(trifluoromethyl)-4′-chlorobenzhydryloxy]azetidine
1-phenylsulfonyl-3-(2,4,4′-trichlorobenzhydryloxy)azetidine
1-(n-butyl)sulfonyl-3-(2,4,4′-trichlorobenzhydryloxy)azetidine
3-[2-(trifluoromethyl)-4′-chlorobenzhydryloxy]-N-(4-chloro-3-nitrophenyl)azetidine-1-carboxamide
3-[2-(trifluoromethyl)-4′-chlorobenzhydryloxy]-N-(2,4-difluorophenyl)azetidine-1-carboxamide
3-[2-(trifluoromethyl)-4′-chlorobenzhydryloxy]-N-(2,3-dihydrobenzo[1,4]dioxin-6-yl)azetidin 1-carboxamide
3-(2,4′-dichlorobenzhydryloxy)-N-(2,6-difluorophenyl)azetidine-1-carboxamide
3-[4-chloro-a-(2-chloropyrid-3-yl)benzyloxy]-N-(1-adamantyl)azetidine-1-carboxamide
3-[2-chloro-a-(2-chloropyridin-5-yl)benzyloxy]-N-(tert-butyl)azetidine-1-carboxamide
3-[(S*)-4-chloro-a-(2-chloropyidin-3-yl)benzyloxy]-N-(tert-butyl)azetidine-1-carboxamide
3-[2-(trifluoromethyl)-4′-chlorobenzhydryloxy]-N-(benzoyl)azetidine-1-carboxamide
3-[2-(trifluoromethyl)-4′-fluorobenzhydryloxy]-N-(benzoyl)azetidine-1-carboxamide
3-(2,4′-dichlorobenzhydryloxy)-N-(piperidyl)azetidine-1-carboxamide
4-(2,4′-dichlorobenzhydryloxy)-N-(1-adamantyl)piperidine-1-carboxamide
1-(1-piperidinecarbonyl)-4-(2,4′-dichlorobenzhydryloxy)piperidine
4-(2,4,4′-trichlorobenzhydryloxy)-N-(tert-butyl)piperidine-1-carboxamide
4-(2,4,4′-trichlorobenzhydryloxy)-N-(cyclohexyl)piperidine-1-carboxamide
4-(2,4,4′-trichlorobenzhydryloxy)-N-methyl-N-benzylpiperidine-1-carboxamide
1-(1-piperidinecarbonyl)-4-(2,4,4′-trichlorobenzhydryloxy)piperidine, and
1-(tert-butylacetyl)-4-(2,4′-dichlorobenzhydryloxy)piperidine.

According to a further aspect of the present invention there is provided a method of treatment of a disorder mediated by CB₁ receptors comprising administration to a subject in need of such treatment an effective dose of the compound with which the invention is concerned, or a pharmaceutically acceptable salt or prodrug thereof.

The disorders mediated by CB₁ receptors are selected from psychosis, memory deficit, cognitive disorders, attention deficit disorder, migraine, neuropathy, neuro-inflammatory disorders including multiple sclerosis and Guillain-Barre syndrome and the inflammatory sequelae of viral encephalitis, cerebral vascular injuries, head trauma, anxiety disorders, depression, stress, epilepsy, dementia, distonia, Alzheimer's disease, Huntingdon's disease, Tourette's syndrome, ischaemia, pain, Parkinson's disease, schizophrenia, substance abuse disorders especially relating to nicotine, alcohol, and opiates, smoking cessation, treatment of nicotine dependance and/or treatment of symptoms of nicotine withdrawal, gastrointestinal disorders (such as dysfunction of gastrointestinal motility or diarrhoea), obesity and other eating disorders associated with excessive food intake, and associated health complications including non-insulin dependant diabetes mellitus.

The present invention is particularly directed to psychosis, memory deficit, cognitive disorders, attention deficit disorder, migraine, anxiety disorders, stress, epilepsy, Parkinson's disease, schizophrenia, substance abuse disorders especially relating to nicotine, alcohol, and opiates, smoking cessation, treatment of nicotine dependance and/or treatment of symptoms of nicotine withdrawal, gastrointestinal disorders (such as dysfunction of gastrointestinal motility or diarrhoea), obesity and other eating disorders associated with excessive food intake, and associated health complications including non-insulin dependant diabetes mellitus.

The present invention is more particularly directed to disorders selected from psychosis, schizophrenia, cognitive disorders, attention deficit disorder, smoking cessation, gastrointestinal disorders (such as dysfunction of gastrointestinal motility or diarrhoea), obesity and other eating disorders associated with excessive food intake (including bulimia and compulsive eating disorder) and associated health complications including non-insulin dependant diabetes mellitus. The present invention is particularly directed to obesity and other eating disorders associated with excessive food intake and associated health complications including non-insulin dependant diabetes mellitus, and particularly to obesity and other eating disorders associated with excessive food intake, and especially to obesity.

In an alternative embodiment, the present invention is directed to substance abuse disorders especially relating to nicotine, alcohol, and opiates, smoking cessation, treatment of nicotine dependance and/or treatment of symptoms of nicotine withdrawal, and particularly to smoking cessation and the facilitation thereof.

In a further alternative embodiment, the present invention is directed to gastrointestinal disorders (such as dysfunction of gastrointestinal motility or diarrhoea).

In a further alternative embodiment, the present invention is directed to the treatment of Parkinson's Disease.

The present invention may be employed in respect of a human or animal subject, more preferably a mammal, more preferably a human subject.

As used herein, the term “treatment” as used herein includes prophylactic treatment.

The compound of formula (I) may be used in combination with one or more additional drugs useful in the treatment of the disorders mentioned above, the components being in the same formulation or in separate formulations for administration simultaneously or sequentially.

In a further aspect the invention provides a method of preparation of the compounds of formula (I). Compounds of formula (I) may be prepared according to the following reaction schemes (P is a nitrogen protecting group; R¹ and R² are as previously described; n and m are as previously stated). According to Reaction Scheme 1, the ether (IV) may be formed by reaction of the alcohol (II) with the alcohol (III) with loss of water (for example azeotropic removal of water under standard acidic Dean-Stark conditions). Formation of the amine (V) may be achieved by reaction of (V) with a suitable nitrogen deprotection agent. For example, if P is a benzyl or benzhydryl group, then deprotection may be carried out by treatment with 1-chloroethyl chloroformate followed by methanol. The deprotected cyclic amine (V) can be isolated directly as the hydrochloride salt or, upon basification, as the free-base. The cyclic amine (V) may be converted to the active carbamoyl chloride intermediate (V) via reaction, for example, with phosgene or triphosgene. Furthermore, when P is a benzyl or benzhydryl group, the protected cyclic amine (IV) may be treated with phosgene to give the carbamoyl chloride (V) directly.

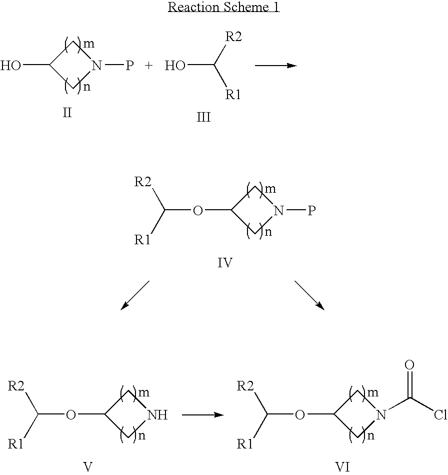

Reaction Scheme 2 details how the intermediate cyclic amine (V) may be converted to final urea (VII) by reaction with an isocyanate (R⁹—N═C═O) or carbamoyl chloride (R⁹NHCOCl). Alternatively, the carbamoyl chloride (VI) may be reacted with an amine (R⁹NH2) to afford the urea (VII). When neither R⁹ nor R¹⁰ is H, the carbamoyl chloride (VI) may be reacted with a secondary amine, R⁹(R¹⁰)NH, to afford the urea (VII). Alternatively, the cyclic amine (V) may be reacted with a carbamoyl chloride, R⁹(R¹⁰)NCOCl, to afford the urea (VIII).

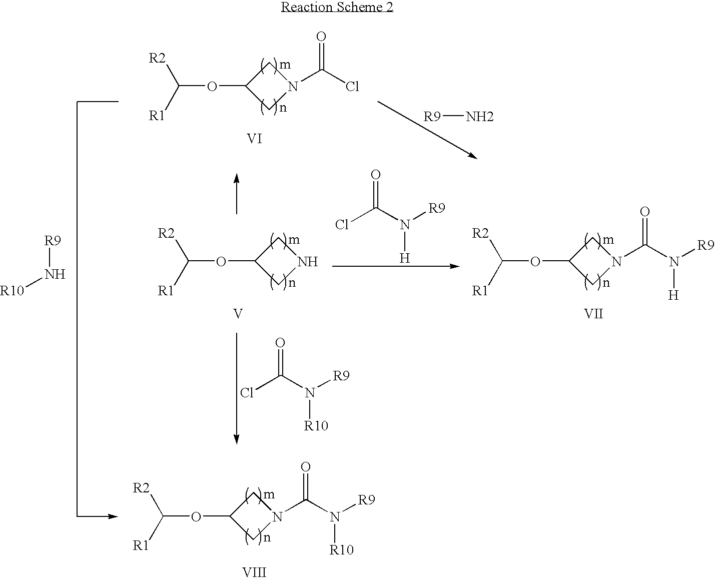

Reaction Scheme 3 details reaction of the carbamoyl chloride (VI) with an alcohol (R¹⁵—OH) to give the final carbamate (IX). The same product may also be formed by reaction of the cyclic amine (V) with a suitable chloroformate (R¹⁵OCOCl).

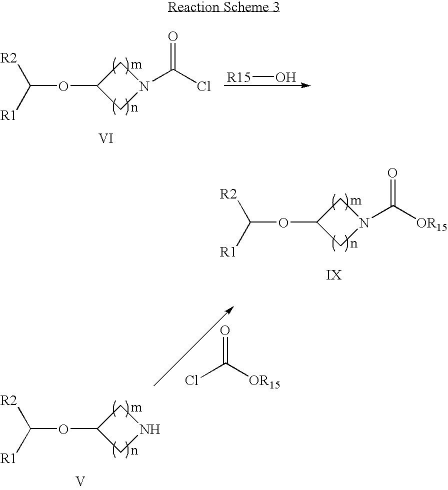

Reaction Scheme 4 shows how the thiourea products (X) may be prepared by reaction of the intermediate cyclic amine (V) with a suitable isothiocyanate reagent (R⁹N═C═S). Furthermore, the cyclic amine (V) may be reacted with, for example, sulfonyl chlorides to afford the sulfonamide products (XI) or with activated carboxylic acid derivatives (for example acid chlorides) to form the amide products (XII).

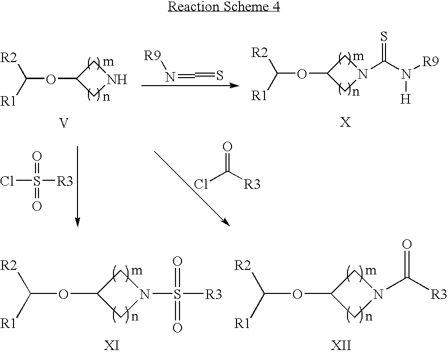

Reaction Scheme 5 details how the cyclic amine intermediates (V) may be alkylated, for example by reaction of a suitable alkyl derivative, R³—[C(R⁷)(R⁸)]ₚ—X (where X is a leaving group such as Br, I, OTs) and a strong base (for example sodium hydride) to give the tertiary amine products (XIII).

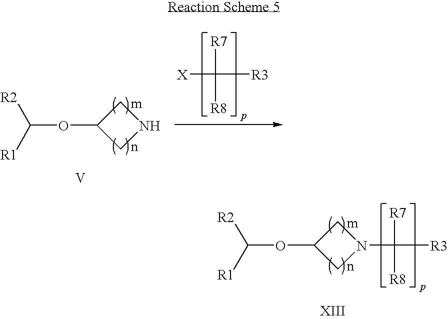

Compounds where R¹¹ and R¹² are not hydrogen can be made by analogous methods.

The invention further provides a pharmaceutical composition comprising an effective amount of the compound of formula (I) in combination with a pharmaceutically acceptable carrier or excipient and a method of making such a composition comprising combining an effective amount of the compound of formula (I) with a pharmaceutically acceptable carrier or excipient.

To further increase efficacy, the composition may contain components such as dextrans or cyclodextrins or ether derivatives thereof, which aid stability and dispersion, and decrease metabolism of the active ingredient.

For compositions in which the pharmaceutically acceptable carrier comprises a cyclodextrin or an ether derivative thereof, the active ingredient is intimately mixed with an aqueous solution of the cyclodextrin or ether derivative thereof, with optional addition of further pharmaceutically acceptable ingredients before, during or after said mixing. The thus obtained solution is optionally lyophilized, and the lyophilized residue is optionally reconstituted with water.

In an embodiment of the present invention, the composition further comprises a buffer system, an isotonizing agent and water.

Compounds of formula (I) may be administered in a form suitable for oral use, for example a tablet, capsule, aqueous or oily solution, suspension or emulsion; for topical use including transmucosal and transdermal use, for example a cream, ointment, gel, aqueous or oil solution or suspension, salve, patch or plaster; for nasal use, for a example a snuff, nasal spray or nasal drops; for vaginal or rectal use, for example a suppository; for administration by inhalation, for example a finely divided powder or a liquid aerosol; for sublingual or buccal use, for example a tablet or capsule; or for parenteral use (including intravenous, subcutaneous, intramuscular, intravascular or infusion), for example a sterile aqueous or oil solution or suspension. In general the above compositions may be prepared in a conventional manner using conventional excipients, using standard techniques well known to those skilled in the art of pharmacy. Preferably, the compound is administered orally.

For oral administration, the compounds of formula (I) will generally be provided in the form of tablets or capsules or as an aqueous solution or suspension.

Tablets for oral use may include the active ingredient mixed with pharmaceutically acceptable excipients such as inert diluents, disintegrating agents, binding agents, lubricating agents, sweetening agents, flavouring agents, colouring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

Capsules for oral use include hard gelatin capsules in which the active ingredient is mixed with a solid diluent, and soft gelatin capsules wherein the active ingredient is mixed with water or an oil such as peanut oil, liquid paraffin or olive oil. Alternatively, the active ingredient may be mixed with excipients, surfactants or solubilising agents such as Labrafil®, Labrasol® or Miglyol®, or appropriate mixtures thereof.

For intramuscular, intraperitoneal, subcutaneous and intravenous use, the compounds of formula (I) will generally be provided in sterile aqueous solutions or suspensions, buffered to an appropriate pH and isotonicity. Suitable aqueous vehicles include Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

It will be appreciated that the dosage levels used may vary over quite a wide range depending upon the compound used, the severity of the symptoms exhibited by the patient and the patient's body weight.

The invention will now be described in detail with reference to the following pharmacological examples. It will be appreciated that the examples are intended to illustrate and not to limit the scope of the present invention.

### EXAMPLES

### Assay Procedures

### Binding to CB₁ Receptors

The binding of compounds of Formula I to recombinant human CB₁ receptors was determined in vitro by standard methods, with reference to the procedure described by Rinaldi-Carmona et al (Rinaldi-Carmona, M., Pialot, F., Congy, C., Redon, B., Barth, F., Bachy, A., Breliere, J. C., Soubre, P., LeFur, G., Life Sci. 1996, 58(15), 1239-1247). Membranes were prepared from HEK293 cells expressing recombinant hCB₁ receptors. Binding assays are performed in a total volume of 250 μL, containing [³H]-SR-141716A (1 nM final concentration), membranes and test compound. Non-specific binding is determined using CP55,940 (10 μM). Serial dilutions are performed starting from test compounds as 10 mM solutions in DMSO. Compounds are tested over the concentration range 100⁻¹⁰ M to 10⁻⁵ M. Kᵢ values are calculated from IC₅₀ values using the Cheng-Prusoff equation.

The thus-determined activity of compounds of formula (D is shown in Table 1.

TABLE 1

| Example | Kᵢ (hCB₁) nM |
|---|---|
| 1 | 0.8 |
| 4 | 1.5 |
| 11 | 3.2 |
| 20 | 5.5 |
| 32 | 27.7 |
| 34 | 8.6 |

### Blockade of □⁹-THC Induced Hypolocomotion in Mice

The in vivo activity of compounds of formula (I) is assayed for ability to antagonise the reduction in locomotor behaviour induced by acute systemic administration of □⁹-THC in male C57B1/6 mice. The procedure was as follows.

Test compounds are assessed following acute oral or intraperitoneal administration at a dose of 30 mg/kg. Each study utilises a between-subjects design (typically n=8) and compares the effects of doses of the test agent to those of vehicle and a positive control.

The route of test compound administration, drug volume and injection-test-interval are dependent upon the compounds used. 10 min before testing, a 3 mg/kg dose □⁹-THC (or vehicle) is administered to mice by the i.p. route. Automated boxes (AM-1052 activity monitors, Benwick Electronics, Linton Instrumentation) are used to record photocell beam breaks as a measure of locomotor activity. The light beams are arranged on a 7 by 4 matrix on a metal grid. 16 grids are connected in series and Perspex boxes, 20 (width)×40 (length)×20 height) cm, with a flat perforated, Perspex lid are placed in each grid. Mice are placed singly in Perspex boxes and the recording of activity in all 16 boxes starts simultaneously. The mice are left undisturbed to explore the novel activity monitor boxes for 15 minutes while beam breaks are recorded.

Locomotor activity data are subjected to one-way analysis of variance (ANOVA) with drug treatment as a between-subjects factor. A significant main effect is followed up by the performance of Dunnett's test in order to assess which treatment mean(s) are significantly different from the control mean. Significant differences between the vehicle/□⁹-THC group and Test compound/□⁹-THC groups are assessed by Newman-Keuls test. All statistical analyses were performed using Statistica Software, Version 6.0 (Statsoft Inc.) and Microsoft Excel 7.0 (Microsoft Corp.).

### Regulation of Feeding Behaviour

The in vivo activity of compounds of formula (1) is assayed for ability to regulate feeding behaviour by measuring food consumption in male food-deprived Lister-hooded rats as follows.

Test compounds are assessed following acute administration. Each study utilises a between-subjects design (typically n=8) and compares the effects of doses of the test agent to those of vehicle and a positive control.

The anorectic drug sibutramine, or the reference CB₁ receptor antagonist, SR-141716A, normally serves as a positive control. The route of drug administration, drug volume and injection-test-interval are dependent upon the compounds used. The injection-test-interval is the time between dosing and food re-presentation. Typically, animals are fasted such that at the time of food re-presentation food has been withdrawn for an 18-hour period. Food consumption is assayed at predetermined time points (typically 1, 2 and 4 hours after administration). Food intake data are subjected to one-way analysis of variance (ANOVA) with drug as a between-subjects factor. A significant main effect is followed up by the performance of Dunnett's test in order to assess which treatment mean(s) are significantly different from the control mean. All statistical analyses were performed using Statistica Software, Version 6.0 (Statsoft Inc.) and Microsoft Excel 7.0 (Microsoft Corp.).

### Analytical Chemistry Procedures

### HPLC

LC (50/80) refers to elution of a sample through an XTERRA RP18 (50 mm×4.6 mm) 5 μm column under gradient conditions. The initial eluent comprises 50% Methanol (pump-A) and 50% of a 10 mM aqueous ammonium acetate solution containing 5% IPA (pump-B) at a flow rate of 2 mL/min. After 1 min, a gradient is run over 5 min to an end point of 80% pump-A and 20% pump-B, which is isocratically maintained for a further 3 min. UV peak detection is generally carried out at a wavelength of 220 nm.

LC (80/20) refers to elution of a sample through an XTERRA RP18 (50 mm×4.6 mm) 5 μm column under isocratic conditions. The eluent comprises 80% Methanol bump-A) and 20% of a 10 mM aqueous ammonium acetate solution containing 5% IPA (pump-B) at a flow rate of 2 mL/min over a period of 10 minutes. UV peak detection is generally carried out at a wavelength of 220 nm.

LC (CHIRAL AD) refers to elution of a sample through a CHIRALPAK AD column (250 mm×4.6 mm) 10 μm column under isocratic conditions. The eluent typically comprises 90% n-hexane and 10% 2-propanol at flow rate of 1 mL/min over a period of 40 minutes. UV peak detection is generally carried out at a wavelength of 220 nm.

### ¹H NMR

Proton nmr spectra were recorded on a 400 MHz Bruker spectrometer. Solutions were typically prepared in either deuterochloroform (CDCl₃) or deuterated dimethylsilfoxide (d⁶-DMSO) with chemical shifts reported in d with reference to tetramethylsilane (TMS) as an internal standard, and coupling constants reported in Hz.

### MS

Mass Spectra were acquired via loop injection on a Waters ZQ Mass Detector equipped with an Electrospray source operated in Positive/Negative Ion switching mode and a cone voltage of 25V.

### Synthetic Examples

### Preparation of 1-benzhydryl-3-azetidinol (1)

This material was prepared according the method of Anderson and Lok (J. Org. Chem., 1972, 37, 3953—the disclosure of which is incorporated herein by reference), m.p. 111-112° C. (lit. m.p. 113° C.).

### Preparation of 2,4′-dichlorobenzhydrol (2)

To a stirred solution of 2,4′-dichlorobenzophenone (239 mmol) in methanol (400 mL) was added sodium borohydride (119 mmol) portionwise at 0° C. The reaction mixture was warmed to room temperature and stirred for 1 hour, then quenched with water and the methanol was removed under reduced pressure. The residue was diluted with dichloromethane (400 mL) and washed with water and brine, dried (MgSO₄) and concentrated in vacuo to yield the product as an orange oil (47.1 g, 78%).

NMR (400 MHz, d⁶-DMSO) δ_{H} 5.99 (1H, d, J=4.5 Hz), 6.16 (1H, d, J=4.5 Hz), 7.35 (7H, m), 7.66 (1H, m)

### Preparation of 1-benzhydryl-3-(2,4′-dichlorobenzhydryloxy)azetidine (3)

A solution of 2,4′-dichlorobenzhydrol (178 mmol), p-toluenesulphonic acid (198 mmol) and 1-benzhydryl-3-azetidinol (99 mmol) in toluene (500 mL) was heated at reflux in a Dean-Stark apparatus for 40 minutes. The solution was cooled, washed with sodium hydrogen carbonate (saturated aqueous solution, 700 mL), dried (MgSO₄) and concentrated in vacuo. The residue was purified by column chromatography [SiO₂; (10% ethyl acetate:isohexane)] to finish the product as a yellow oil (17.8 g, 38%).

NMR (400 MHz, d⁶-DMSO) δ_{H} 2.78 (1H, t, J=6.5 Hz), 2.82 (1H, t, J=6.5 Hz), 3.24 (1H, t, J=5.5 Hz), 3.28 (1H, t, J=5.5 Hz), 4.17 (1H, m), 4.39 (1H, s), 5.74 (1H, s), 7.16 (2H, m), 7.25 (4H, m), 7.32 (3H, m), 7.37 (8H, m), 7.57 (1H, m).

### Preparation of 3-(2,4′-dichlorobenzhydryloxy)azetidine hydrochloride (4)

To a stirred solution of 1-benzhydryl-3-(2,4′-dichlorobenzhydryloxy)azetidine (3) (38 mmol) in dichloromethane (400 mL) was added 1-chloroethyl chloroformate (94 mmol) dropwise at 0° C. and the reaction mixture stirred at room temperature overnight. The reaction mixture was concentrated in vacuo, then dissolved in methanol (400 mL) and stirred at room temperature for 2 hours. The reaction mixture was reduced in vacuo and diluted with isohexane (20 mL), diisopropyl ether (200 mL) and methanol (30 mL) to reveal the product as a white solid (10.1 g, 77% Yo).

NMR (400 MHz, d⁶-DMSO) δ_{H} 3.86 (2H, br d), 4.02 (2H, br d), 4.43 (1H, quintet, J 6.0 Hz) 5.86 (1H, s), 7.37 (3H, m), 7.44 (4H, m), 7.61 (1H, m), 8.98 (2H, br d)

### Preparation of 3-(2,4′-dichlorobenzhydryloxy)azetidine-1-carbonyl chloride (5)

3-(2,4′-Dichlorobenzhydryloxy)azetidine hydrochloride (4) was converted to the corresponding free-base using standard methods. To a stirred solution of 3-(2,4′-dichlorobenzhydryloxy)azetidine (11 mmol) in anhydrous dichloromethane at ice bath temperature was added phosgene (20% in toluene; 13 mmol) dropwise. The reaction was stirred at room temperature for 20 minutes and reduced in vacuo to afford a yellow oil, which was used immediately without purification.

### Example 1

### 4-[3-(2,4′-dichlorobenzhydryloxy)azetidine-1-carbonyl]-[1,4]diazepine-1-carboxylic acid tert-butyl ester (6)

A solution of 3-(2,4′-dichlorobenzhydryloxy)azetidine-1-carbonyl chloride (5) (0.540 mmol), tert-butyl-1,4-diazapine-1-carboxylate (0.810 mmol), and MP-carbonate (3.10 mmol/g, 1.62 mmol) in anhydrous dichloromethane (3 mL) was shaken at room temperature overnight. The reaction mixture was eluted through a pre-wet (dichloromethane) SCX-2 (2 g) cartridge with dichloromethane to yield a colourless gum (48.2 mg, 16%).

MS 534 [M+H]⁺

LC (80/20) 90.0%, 1.10 min

### Example 2

### 3-(2,4′-dichlorobenzhydryloxy)-N-methyl-N-pentyl-azetidine-1-carboxamide (7)

This material was prepared from the corresponding amine by the method described for 4-[3-(2,4′-dichlorobenzhydryloxy)azetidine-1-carbonyl]-[1,4]diazepine-1-carboxylic acid tert-butyl ester (6) (160 mg, 63%).

MS 435 [M+H]⁺

LC (80/20) 99.4%, 1.56 min

### Preparation of 2,4,4′-trichlorobenzhydrol (8)

To a stirred solution of 2,4-dichlorobenzaldehyde (286 mmol) in diethyl ether (500 mL) was added 4-chlorophenylmagnesium bromide (1.0M in diethyl ether, 286 mmol) dropwise at 0° C. over a period of 1 hour. The reaction mixture was warmed to room temperature and stirred for 3 hours. The reaction mixture was quenched with saturated ammonium chloride solution (500 mL) and extracted with diethyl ether (2×500 mL), the extracts were washed with water and brine, dried (MgSO₄) and concentrated in vacuo. The residue was purified by trituration with isohexane (500 mL) to yield the product as a white solid (60.0 g, 73%).

NMR (400 MHz, d⁶-DMSO) δ_{H} 5.95 (1H, d, J=4.5 Hz), 6.23 (1H, d, J=4.0 Hz), 7.31 (2H, m), 7.37 (2H, m), 7.47 (1H, dd, J 2.5, 8.5 Hz), 7.55 (1H, d, J=2.0 Hz), 7.66 (1H, d, J=8.5 Hz).

### Preparation of 1-benzhydryl-3-(2,4,4′-trichlorobenzhydryloxy)azetidine (9)

A solution of 2,4,4′-trichlorobenzhydrol (8) (174 mmol), p-toluenesulphonic acid (174 mmol) and 1-benzhydryl-3-azetidinol (1) (87 mmol) in toluene (700 mL) was heated at reflux under Dean-Stark conditions for 30 minutes. The solution was cooled, washed with sodium hydrogen carbonate (saturated aqueous solution, 700 mL), dried (MgSO₄) and concentrated in vacuo. The residue was purified by column chromatography [SiO₂; (10% ethyl acetate:isohexane)] to furnish the product as a yellow oil (22.0 g, 50%).

NMR (400 MHz, d⁶-DMSO) δ_{H} 2.78 (1H, t, J=6.5 Hz), 2.84 (1H, t, J=6.5 Hz), 3.23 (1H, t, J=6.5 Hz), 3.31 (1H, t, J=6.5 Hz), 4.18 (1H, m), 4.39 (1H, s), 5.71 (1H, s), 7.15 (2H, m), 7.24 (4H, m), 7.32 (2H, m), 7.37 (6H, m), 7.47 (1H, m), 7.57 (2H, m).

### Preparation of 3-(2,4,4′-trichlorobenzhydryloxy)azetidine (10)

To a stirred solution of 1-benzhydryl-3-(2,4,4′-trichlorobenzhydryloxy)azetidine (9) (39 mmol) in dichloromethane (400 mL) was added 1-chloroethyl chloroformate (98 mmol) dropwise at 0° C. and the reaction mixture stirred at room temperature overnight. The reaction mixture was concentrated in vacuo, then dissolved in methanol (400 mL) and stirred at room temperature for 1 hour. The reaction mixture was concentrated in vacuo, then diluted with ethyl acetate (400 mL) and washed with sodium hydroxide (5N, 400 mL), dried (MgSO₄) and concentrated in vacuo to finish a yellow oil. The residue was purified by filtration through silica, eluting with dichloromethane, then [ethyl acetate:methanol:ammonium hydroxide (90:8:2)] to yield a yellow oil (8.0 g, 60%). NMR (400 MHz, d⁶-DMSO) δ_{H} 3.38 (4H, br d), 4.29 (1H, m), 5.68 (1H, s), 7.32 (2H, m), 7.40 (2H, m), 7.49 (2H, m), 7.50 (1H, m), 7.60 (2H, m)

### Preparation of 3-(2,4,4′-trichlorobenzhydryloxy)azetidine-1-carbonyl chloride (11)

This material was prepared from 3-(2,4,4′-trichlorobenzhydryloxy)azetidine (10) using the procedure described for 3-(2,4′-Dichlorobenzhydryloxy)azetidine-1-carbonyl chloride (5).

### Example 3

### 3-(2,4,4′-trichlorobenzhydryloxy)N-methyl-N-benzyl-azetidine-1-carboxamide (12)

This compound was prepared from 3-(2,4,4′-trichlorobenzhydryloxy)azetidine-1-carbonyl chloride (11) and N-methylbenzylamine using the procedure described for 4-[3-(2,4′-dichlorobenzhydryloxy)azetidine-1-carbonyl]-[1,4]diazepine-1-carboxylic acid tert-butyl ester (6) (183 mg, 69%).

MS 489 [M+H]⁺

LC (50/80) 99.9%, 8.07 min (50/80)

### Preparation of 2-(trifluoromethyl)-4′-chlorobenzhydrol (13)

Magnesium turnings (4.21 g, 170 mmol) were stirred under nitrogen for 10 min. Stirring was halted and a solution of 2-bromobenzotrifluoride (36.37 g, 160 mmol) in dry THF (160 mL) was added via a dropping funnel until the magnesium turnings were just covered. The reaction mixture was heated with a hot-air gun until localised turbidity was observed. At this point, stirring was initiated, and the rate of the reaction was subsequently controlled with intermittent use of an ice-water bath, and varying the rate of addition of the remaining 2-bromobenzotrifluoride solution. After complete addition, the mixture was allowed to stir for 1 h, then used as a ˜0.9 M solution.

To a stirred solution of 4-chlorobenzaldehyde (2.17 g, 15 mmol) in anhydrous THF (10 mL) was added a solution of 2-(trifluoromethyl)phenylmagnesium bromide (˜0.9 M; 18 mL, 16 mmol) over 2 min. After 16 h, the resultant mixture was partitioned between diethyl ether and 1N HCl. The aqueous phase was extracted with diethyl ether (2×30 mL) and the combined organic extracts were washed with 1N HCl, brine and dried (MgSO₄). Evaporation under reduced pressure afforded the desired product as an amber oil (4.63 g, 100%).

MS 269 [M-OH]⁺

LC (50/80) 97.6%, 5.47 min

### Preparation of 1-benzhydryl-3-[2-(trifluoromethyl)-4′-chlorobenzhydryloxy]azetidine (14)

This material was prepared from 1-benzhydryl-3-azetidinol (1) (40.1 mmol) and 2-(trifluoromethyl)-4-chlorobenzhydrol (13) (80.2 mmol) using the procedure described for 1-benzhydryl-3-(2,4,4′-trichlorobenzhydryloxy)azetidine (9) (13.5 g, 66%). NMR (400 MHz, d⁶-DMSO) δ_{H} 2.74 (1H, br t), 2.86 (1H, br t), 3.20 (1H, br t), 3.29 (1H, br t), 4.15 (1H, q, J=6.0 Hz), 4.39 (1H, s), 5.71 (1H, s), 7.16 (2H, m), 7.26 (6H, m), 7.38 (6H, m), 7.54 (1H, m), 7.70 (3H, m).

### Preparation of 3-[2-(trifluoromethyl)-4′-chlorobenzhydryloxy]azetidine hydrochloride (15)

This material was prepared from 1-benzhydryl-3-[2-(trifluoromethyl)-4′-chlorobenzhydryloxy]azetidine (14) (25.6 mmol) using the procedure described for 3-(2,4′-dichlorobenzhydryloxy)azetidine hydrochloride (4) (8.2 g, 85%).

NMR (400 MHz, d⁶-DMSO) δ_{H} 3.78 (1H, m), 3.97 (3H, m), 4.89 (1H, q, J=6.0 Hz), 5.85 (1H, s), 7.33 (2H, m), 7.44 (2H, m), 7.59 (1H, m), 7.76 (3H, m), 8.97 (1H, br s)

### Example 4

### 3-[2-(trifluoromethyl)-4′-chlorobenzhydryloxy]-N-methyl-N-benzyl-azetidine-1-carboxamide (16)

3-[2-(trifluoromethyl)-4′-chlorobenzhydryloxy]azetidine hydrochloride (15) was converted to the corresponding free-base by partitioning between dichloromethane and 0.5N sodium hydroxide.

To a stirred solution of triphosgene (187 mg, 0.63 mmol) in dry dichloromethane (5 mL) at 0° C. was added portionwise over 40 min a solution of the free-base of compound (15) (1.66 mmol) in dry dichloromethane (10 mL). After 20 min, a 5 mL aliquot of the reaction mixture was rapidly added to triethylamine (230 □L) and N-methylbenzylamine (73 □L) and the resultant mixture was shaken at ambient temperature for 72 h. The reaction mixture was washed with 0.2N hydrochloric acid and the organics were loaded onto an SCX-2 (2 g) cartridge, then eluted with DCM followed by EtOAc. The combined organics were concentrated in vacuo, then purified by flash column chromatography [SiO₂; ethyl acetate:iso-hexane (30:70-35:65)] to afford the desired product (181 mg, 67%).

MS489[M+H]⁺

LC (50/80) 96.9%, 7.60 min

### Preparation of 2-methyl-4′-chlorobenzhydrol (17)

This compound was prepared from 2-tolualdehyde (15 mmol) and 4-chlorophenylmagnesium bromide (16 mmol) using the same procedure described for 2,4,4′-trichlorobenzhydrol (8) (2.97 g, 85%)

NMR (400 MHz, d⁶-DMSO) δ_{H} 2.20 (3H, s), 5.83 (2H, s), 7.08-7.21 (3H, m), 7.26-7.42 (5H, m)

### Preparation of 1-benzhydryl-3-(2-methyl-4′-chlorobenzhydryloxy)azetidine (18)

This material was prepared from 2-methyl-4′-chlorobenzhydrol (17) (12.7 mmol) using the procedure described for 1-benzhydryl-3-(2,4,4′-trichlorobenzhydryloxy)azetidine (9). Flash column chromatography [SiO₂; iso-hexane—ethyl acetate (12:1)] afforded the desired material as a yellow oil (0.87 g, 30%)

MS 455 [M+H]⁺

LC (50/80) 93%, 2.07 min

### Preparation of 3-(2-methyl-4′-chlorobenzhydryloxy)azetidine hydrochloride (19)

This material was prepared from 1-benzhydryl-3-(2-methyl-4′-chlorobenzhydryloxy)azetidine (18) (1.87 mmol) using the procedure described for 3-(2,4′-Dichlorobenzhydryloxy)azetidine hydrochloride (4). Trituration with diethyl ether afforded the required product as a white solid (0.44 g, 72%).

MS 288 [M+H]⁺

LC (50/80) 99.0%, 2.69 min

### Preparation of 3-(2-methyl-4′-chlorobenzhydryloxy)azetidine-1-carbonyl chloride (20)

This material was prepared from 3-2-methyl-4′-chlorobenzhydryloxy)azetidine hydrochloride (19) using the procedure described for 3-(2,4′-dichlorobenzhydryloxy)azetidine-1-carbonyl chloride (5).

### Example 5

### 3-(2-methyl-4′-chlorobenzhydryloxy)-N-methyl-N-benzyl-azetidine-1-carboxamide (21)

This compound was prepared from 3-(2-methyl-4′-chlorobenzhydryloxy)azetidine-1-carbonyl chloride (20) using the procedure described for 4-[3-(2,4′-dichlorobenzhydryloxy)azetidine-1-carbonyl]-[1,4]diazepine-1-carboxylic acid tert-butyl ester (6).

MS 435 [M+H]⁺

LC (50/80) 93.0%, 7.43 min

### Example 6

### 3-(2,4′-dichlorobenzhydryloxy)-N-(tert-butyl)-azetidine-1-thiocarboxamide (22)

A mixture of 3-(2,4′-dichlorobenzhydryloxy)azetidine hydrochloride (4) (200 mg, 0.58 mmol), MP-carbonate (3.01 mmol/g; 578 mg, 1.74 mmol), tert-butyl isothiocyanate (74 □L, 0.58 mmol) and dichloromethane (5 mL) was shaken at ambient temperature for 72 h. The mixture was loaded onto an SCX-2 (2 g) cartridge, which was eluted with dichloromethane (20 mL). Evaporation afforde a crude oil that was purified by flash column chromatography [SiO₂; ethyl acetate—iso-hexane (1:1)] to give the desired product (215 mg, 88%)

NMR (400 MHz, d⁶-DMSO) δ_{H} 1.42 (9H, s), 3.78 (1H, ddd, J 10.0, 4.0, 1.0 Hz), 3.83 (1H, ddd, J 10.0, 4.0, 1.0 Hz), 4.06 (1H, ddd, J 10.0, 6.0, 1.0 Hz), 4.12 (1H, ddd, J 10.0, 6.0, 1.0 Hz), 4.26-4.32 (1H, m), 5.79 (1H, br s), 6.36 (1H, br s), 7.32-7.38 (3H, m), 7.40-7.46 (4H, m), 7.62 (1H, dd, J 8.2 Hz)

LC (50/80) 99.4%, 7.70 min

### Example 7

### 3-[(R*)-2,4′-dichlorobenzhydryloxy]-N-(tert-butyl)-azetidine-1-thiocarboxamide (23); and

### Example 8

### 3-[(S*)-2,41-dichlorobenzhydryloxy]-N-(tert-butyl)-azetidine-1-thiocarboxamide (24)

The racemic compound 3-(2,4′-dichlorobenzhydryloxy)-N-(tert-butyl)azetidine-1-thiocarboxamide (22) was separated into samples which were significantly enriched in each single enantiomeric form. Thus, a sample of the racemic mixture (52.4 mg) was dissolved in 1 mL of an IPA—hexane (10:90) mix. This mixture was injected (900 □L) onto a Daicel Chiralpak® AD™ chiral HPLC column (250 mm×21 mm ID) fitted with a guard column (50 mm×21 mm ID) [eluent: IPA—hexane (10:90); flow-rate: 10 mL/min; wavelength: 235 nm]. The combined first-eluting enantiomer (arbritrarily assigned R* unknown absolute stereochemistry) fractions were evaporated to afford 3-[(R*)-2,4′-dichlorobenzhydryloxy]-N-(tert-butyl)azetidine-1-thiocarboxamide (17.6 mg, 37.3%).

LC [Chiral AD; IPA—hexane (5:95)] 91.9%, 20.51 min; 0.5%, 24.04 min; 99% ee

The combined second-eluting enantiomer (arbritrarily assigned S* unknown absolute stereochemistry) fractions were evaporated to afford 3-[(5*)-2,4′-dichlorobenzhydryloxy]-N-(tert-butyl)azetidine-1-thiocarboxamide (15.0 mg, 31.8%).

LC [Chiral AD; IPA—hexane (5:95)] 84.7%, 23.65 min; 12.7%, 20.62 min; 74% ee

### Example 9

### 3-[2-(trifluoromethyl)-4′-chlorobenzhydryloxy]-N-(tert-butyl)-azetidine-1-thiocarboxamide (25)

To a solution of 3-[2-(trifluoromethyl)-4′-chlorobenzhydryloxy]azetidine hydrochloride (15) (100 mg, 0.28 mmol) in anhydrous DCM (3 mL) was added MP-carbonate (3.01 mmol/g; 275 mg, 0.84 mmol) and tert-butyl isothiocyanate (35 □L, 0.28 mmol). The resultant mixture was shaken at ambient temperature for 16 h, after which time it was poured onto a DCM-wetted SCX-2 (1 g) cartridge. The sample was eluted with DCM (18 mL), then evaporated to afford the desired product (112 mg, 89%).

MS 457 [M+H]⁺

LC (50/80) 99.0%, 7.66 min

### Example 10

### 3-[2-(trifluoromethyl)-4′-chlorobenzhydryloxy]-N-(tetrahydrofuran-2-yl-methyl)-azetidine-1-thiocarboxamide (26)

To a solution of 3-[2-(trifluoromethyl)-4′-chlorobenzhydryloxy]azetidine hydrochloride (15) (0.264 mmol) in dichloromethane (3 mL) was added tetrahydrofurfuryl isothiocyanate (0.264 mmol) and triethylamine (0.580 mmol) and the reaction mixture was shaken at room temperature overnight. The reaction mixture was eluted through a pre-wet (CH₂Cl₂) SCX-2 (2 g) cartridge with dichloromethane to yield a colourless gum (10.5 mg, 82%).

NMR (400 MHz, DMSO) δ_{H} 1.53 (1H, m), 1.79 (3H, m), 3.42 (1H, m), 3.59 (1H, m), 3.73 (2H, m), 3.84 (1H, m), 3.95 (1H, m), 4.07 (2H, m), 4.36 (1H, m), 5.78 (1H, s), 7.35 (2H, m), 7.42 (2H, m), 7.56 (2H, m), 7.76 (3H, m)

LC (50/80) 99.8%, 7.01 min

### Example 11

### 3-(2,4,4′-trichlorobenzhydryloxy)-1-[(S)-2-methoxy-2-phenylethanoyl]azetidine (27)

To a mixture of CDI (165 mg, 1 mmol) and (S)-(+)-methoxyphenylacetic acid (167 mg, 1 mmol) was added anhydrous THF (5 mL) and the resultant solution was stirred at ambient temperature. After 1 h, an opaque solution of 3-(2,4,4′-trichlorobenzhydryloxy)azetidine (10) (343 mg, 1 mmol) in THF (5 mL) was added and the mixture was stirred for 16 h. The reaction was partitioned between EtOAc and 0.25 N HCl, the layers separated, and the aqueous phase was extracted with EtOAc. The combined organic extracts were washed consecutively with 0.25 N HCl, water and brine, then dried (MgSO₄). Evaporation afforded a colourless gum which was purified by flash column chromatography [SiO₂; ethyl acetate—iso-hexane (45:55 to 60:40)] to give the desired product as a colourless oil (260 mg, 53%).

NMR (400 MHz, CDCl₃) OH 3.36-3.44 (3H, m), 3.83-4.18 (3H, m), 4.20-4.37 (2H, m), 4.69-4.74 (1H, m), 5.62-5.68 (1H, m), 7.16-7.45 (12H, m)

LC (50/80) 98.0%, 7.76 min

### Example 12

### 3-(2,4′-dichlorobenzhydryloxy)-1-(3-chlorothiophen-2-yl-formyl)azetidine (28)

A solution of 3-(2,4′-dichlorobenzhydryloxy)azetidine hydrochloride (4) (0.58 mmol), 3-chlorothiophene-2-carbonyl chloride (0.58 mmol) and MP-carbonate (3.10 mmol/g, 1.74 mmol) in dichloromethane (3 mL) was shaken at room temperature for overnight. The reaction mixture was eluted through a pre-wet (CH₂Cl₂) SCX-2 (2 g) cartridge with dichloromethane to yield a colourless gum (165 mg, 63%).

NMR (400 MHz, d⁶-DMSO) δ_{H} 4.03 (2H, br s), 4.26 (2H, br d), 4.45 (1H, m), 4.84 (1H, s), 7.13 (1H, m), 7.40 (7H, m), 7.62 (1H, m), 7.85 (1H, m)

LC (50/80) 98.4%, 7.52 min

### Preparation of 2-(trifluoromethyl)-D-(2,3-dihydrobenzo[1,4]dioxin-6-yl)benzyl Alcohol (29)

This material was prepared from 1,4-benzodioxan-6-carboxaldehyde (15 mmol) using the procedure described for 2-(trifluoromethyl)-4′-chlorobenzhydrol (13) (4.52 g, 100%).

### Preparation of 1-benzhydryl-3-[2-(trifluoromethyl)-□-(2,3-dihydrobenzo[1,4]dioxin-6-yl)benzyloxy]azetidine (30)

This material was prepared from 2-(trifluoromethyl)-□-(2,3-dihydrobenzo[1,4]dioxin-6-yl)benzyl alcohol (29) (14.60 mmol) using the procedure described for 1-benzhydryl-3-(2,4′-dichlorobenzhydryloxy)azetidine (3). The product was used directly in the next step without purification.

### Preparation of 3-[2-(trifluoromethyl)-□-(2,3-dihydrobenzo[1,4]dioxin-6-yl)benzyloxy]azetidine (31)

To a stirred solution of (30) (7.30 mmol) in dichloromethane (40 mL) was added 1-chloroethylchloroformate (14.6 mmol) dropwise at 0° C. and the reaction mixture stirred at room temperature overnight. The reaction mixture was concentrated in vacuo, then dissolved in methanol (40 mL) and stirred at room temperature for 1 hour. The reaction mixture was concentrated in vacuo, then diluted with ethyl acetate (400 mL) and washed with sodium hydroxide (5N, 400 mL), dried (MgSO₄) and concentrated in vacuo to furnish a yellow oil. The residue was purified by filtration through silica, eluting with dichloromethane, then ethyl acetate:methanol:ammonium hydroxide (90:8:2) to yield the desired product as a yellow oil (790 mg, 52%).

MS 366 [M+H]⁺

LC (50/80) 87.2%, 2.63 min

### Example 13

### 1-[2-(tert-butyl)acetyl]-3-[2-(trifluoromethyl)-D-(2,3-dihydrobenzo[1,4]dioxin-6-yl)benzyloxy]azetidine (32)

A solution of 3-[2-(trifluoromethyl)-□-(2,3-dihydrobenzo[1,4]dioxin-6-yl)benzloxy]azetidine (31) (0.274 mmol), tert-butyl acetyl chloride (0.274 mmol) and MP-carbonate (3.10 mmol/g; 0.822 mmol) in dichloromethane (3 mL) was shaken at room temperature overnight. The reaction mixture was eluted through a pre-wet (CH₂Cl₂) SCX-2 (2 g) cartridge with dichloromethane and evaporated to yield the desired product as a colourless gum (125 mg, 99%).

MS 464 [M+H]⁺

LC (50/80) 88.3%, 6.61 min

### Preparation of 1-benzhydryl-3-(4,4′-dichlorobenzhydryloxy)azetidine (33)

A solution of 1-benzhydryl-3-azetidinol (1) (45.0 mmol), p-toluenesulphonic acid (90.0 mmol) and 4,4′-dichlorobenzhydrol (90.0 mmol) in toluene (500 mL) was heated at reflux under Dean-Stark conditions for 30 minutes. The solution was cooled, washed with sodium hydrogen carbonate (saturated aqueous solution, 700 mL), dried (MgSO₄) and concentrated in vacuo. The residue was purified by column chromatography [SiO₂; (10% ethyl acetate:isohexane)] to furnish the product as a yellow oil (8.6 g, 40%).

NMR (400 MHz, d⁶-DMSO) δ_{H} 2.80 (2H, m), 3.25 (2H, m), 4.13 (1H, t, J=6.0 Hz), 4.39 (1H, s), 5.50 (1H, s), 7.15 (2H, m), 7.25 (4H, m), 7.31-7.40 (12H, m).

LC (50/80) 99.7%, 3.18 min

### Preparation of 3-(4,4′-dichlorobenzhydryloxy)azetidine Hydrochloride (34)

To a stirred solution of 1-benzhydryl-3-(4,4′-dichlorobenzhydryloxy)azetidine (33) (8.4 mmol) in dichloromethane (50 mL) was added 1-chloroethyl chloroformate (16.9 mmol) dropwise at 0° C. and the reaction mixture stirred at reflux for 6 hours. The reaction mixture was cooled to room temperature, concentrated in vacuo, then dissolved in methanol (50 mL) and stirred at room temperature for overnight. The reaction mixture was reduced in vacuo and triturated with diethyl ether (100 mL) to reveal the product as a white solid (2.16 g, 74%).

NMR (400 MHz, d⁶-DMSO) δ_{H} 3.84 (2H, m), 3.99 (2H, m), 4.39 (1H, quintet, J=6.5 Hz), 5.65 (1H, s), 7.37 (4H, m), 7.44 (4H, m).

LC (50/80) 98.8%, 4.32 min

### Example 14

### 1-cyclopentoyl-3-(4,4′-dichlorobenzhydryloxy)azetidine (35)

This material was prepared from 3-(4,4′-dichlorobenzhydryloxy)azetidine hydrochloride (34) (0.325 mmol) and cyclopentyl carbonyl chloride (0.270 mmol) using the procedure described for compound 28 (77.0 mg, 7%).

MS 404 [M+H]⁺

NMR (400 MHz, CDCl₃) δ_{H} 1.47-1.81 (8H, br m), 3.84 (1H, m), 4.05 (2H, m), 4.18 (1H, m), 4.36 (1H, m), 5.30 (1H, s), 7.20-7.36 (8H, m)

### Example 15

### 1-(4-pyridylformyl)-3-(2,4′-dichlorobenzhydryloxy)azetidine (36)

A solution of 3-(2,4′-dichlorobenzhydryloxy)azetidine hydrochloride (4) (0.19 mmol), 3-nicotinoyl chloride hydrochloride (0.21 mmol) and triethylamine (0.42 mmol) in dichloromethane (2 mL) was shaken at room temperature overnight. The reaction mixture was washed with water, dried (MgSO₄) and condensed to give a crude oil. Flash column chromatography (SiO₂; ethyl acetate) afforded the desired product as a colourless oil (45 mg, 57%).

MS 414 [M+H]⁺

LC (50/80) 99.0%, 5.67 min

### Example 16

### 1-(4-nitrophenylsulfonyl)-3-[2-(trifluoromethyl)-4′-chlorobenzhydryloxy]azetidine (37)

A solution of 3-[2-(trifluoromethyl)-4′-chlorobenzhydryloxy]azetidine hydrochloride (15) (0.264 mmol), 4-nitrophenylsulfonyl chloride (0.264 mmol) and MP-carbonate (2.62 mmol/g; 0.792 mmol) in anhydrous dichloromethane (3 mL) was shaken at room temperature overnight. AP-trisamine (2.49 mmol/g; 0.792 mmol) was added and the reaction was shaken for 2 hours. The reaction mixture was eluted through a pre-wet (CH₂Cl₂) SCX-2 (2 g) cartridge with dichloromethane and evaporated to afford the desired product as a colourless gum (107 mg, 78%).

MS 528 [M+H]⁺

LC (50/80) 98.7%, 7.68 min

### Example 17

### 1-(thiophen-3-ylsulfonyl)-3-[2-(trifluoromethyl)-4′-chlorobenzhydryloxy]azetidine (38)

This material was prepared from 3-[2-(trifluoromethyl)-4′-chlorobenzhydryloxy]azetidine hydrochloride (15) and 3-thiophenyl sulfonyl chloride using the procedure described for compound (37) (107 mg, 85%).

MS 488 [M+H]⁺

LC (50/80) 99.3%, 7.22 min

### Example 18

### 1-(3-fluorophenylsulfonyl)-3-[2-(trifluoromethyl)-4′-chlorobenzhydryloxylazetidine (39)

This material was prepared from 3-[2-(trifluoromethyl)-4′-chlorobenzhydryloxy]azetidine hydrochloride (15) and 3-fluorophenylsulfonyl chloride using the procedure described for compound (37) (95 mg, 73%).

MS 500 [M+H]⁺

LC (50/80) 97.8%, 7.68 min

### Example 19

### 1-phenylsulfonyl-3-(2,4,4′-trichlorobenzhydryloxy)azetidine (40)

A solution of 3-(2,4,4′-trichlorobenzhydryloxy)azetidine (10) (0.292 mmol), phenyl sulfonyl chloride (0.292 mmol) and MP-carbonate (3.01 mmol/g, 0.584 mmol) in anhydrous dichloromethane (3 mL) was shaken at room temperature overnight. The reaction mixture was eluted through a pre-wet (CH₂Cl₂) SCX-2 (2 g) cartridge with dichloromethane and evaporated to yield the desired product as a colourless gum (73 mg, 52%).

NMR (400 MHz, d⁶-DMSO) δ_{H} 3.43 (1H, m), 3.53 (1H, m), 3.83 (1H, m), 3.94 (1H, m), 4.20 (1H, m), 5.61 (1H, s), 7.12 (2H, m), 7.31 (1H, m), 7.37 (2H, m), 7.45 (1H, m), 7.57 (1H, m), 7.69 (2H, m), 7.80 (3H, m)

LC (50/80) 99.8%, 7.80 min

### Example 20

### 1-(n-butyl)sulfonyl-3-(2,4,4′-trichlorobenzhydryloxy)azetidine (41)

This material was prepared from 3-(2,4,4′-trichlorobenzhydryloxy)azetidine (10) and butylsulfonyl chloride using the procedure described for compound (40) (55.3 mg, 55%).

NMR (400 MHz, d⁶-DMSO) δ_{H} 0.87 (3H, t, J=7.5 Hz), 1.370 (2H, m), 1.60 (2H, m), 3.08 (2H, m), 3.72 (1H, m), 3.82 (2H, m), 3.95 (1H, m), 4.39 (1H, m), 5.75 (1H, s), 7.35 (2H, m), 7.43 (2H, m), 7.52 (1H, m), 7.64 (2H, m)

LC (50/80) 93.0%, 7.96 min

### Example 21

### 3-(4,4′-dichlorobenzhydryloxy)-O-(tert-butyl)azetidine-1-carboxylate (42)

A solution of 3-(4,4′-dichlorobenzhydryloxy)azetidine hydrochloride (34) (0.29 mmol), di-tert-butyl dicarbonate (0.32 mmol) and MP-carbonate (3.31 mmol/g, 0.87 mmol) in dichloromethane (3 mL) was shaken at room temperature overnight. The reaction mixture was eluted through a pre-wet (CH₂Cl₂) SCX-2 (2 g) cartridge with dichloromethane to yield the desired product as a colourless gum (73.4 mg, 62%).

NMR (400 MHz, d⁶-DMSO) δ_{H} 1.35 (9H, s), 3.69 (2H, m), 3.93 (2H, m), 4.25 (1H, m), 5.27 (1H, s), 7.38 (4H, m), 7.43 (4H, m)

LC (50/80) 99.3%, 8.00 min

### Example 22

### 3-(4,4′-dichlorobenzhydryloxy)-O-(phenyl)azetidine-1-carboxylate (43)

This material was prepared from 3-(4,4′-dichlorobenzhydryloxy)azetidine hydrochloride (34) and phenyl chloroformate using the procedure described for compound (42) (56 mg, 45%).

NMR (400 MHz, d⁶-DMSO) δ_{H} 3.84 (1H, br s), 3.98-4.15 (2H, br m), 4.27 (1H, br s), 4.41 (1H, m), 5.64 (1H, s), 7.13 (2H, m), 7.20 (1H, m), 7.35-7.46 (9H, m)

LC (50/80) 96.3%, 7.90 min

### Example 23

### 3-(4,4′-dichlorobenzhydryloxy)-O-(benzyl)azetidine-1-carboxylate (44)

This material was prepared from 3-(4,4′-dichlorobenzhydryloxy)azetidine hydrochloride (34) and benzyl chloroformate using the procedure described for compound (42) (84 mg, 65%).

NMR (400 MHz, d⁶-DMSO) δ_{H} 3.79 (2H, br s), 4.04 (2H, br s), 4.32 (1H, m), 5.02 (1H, s), 5.58 (1H, s), 7.29-7.43 (13H, m)

LC (50/80) 97.9%, 8.30 min

### Example 24

### 3-(4,4′-dichlorobenzhydryloxy)-O-(iso-butyl)azetidine-1-carboxylate (45)

This material was prepared from 3-(4,4′-dichlorobenzhydryloxy)azetidine hydrochloride (34) and iso-butyl chloroformate using the procedure described for compound (42) (75 mg, 63%).

NMR (400 MHz, d⁶-DMSO) δ_{H} 0.84 (6H, d, J=7.0 Hz), 1.81 (1H, m), 3.75 (2H, br s), 4.01 (2H, br s), 4.32 (1H, m), 5.58 (1H, s), 7.34-7.43 (8H, m)

LC (50/80) 98.8%, 8.20 min

### Example 25

### 3-[2-(trifluoromethyl)-4′-chlorobenzhydryloxy]-N-(4-chloro-3-nitrophenyl)azetidine-1-carboxamide (46)

A solution of 3-[2-(trifluoromethyl)-4′-chlorobenzhydryloxy)azetidine hydrochloride (15) (0.264 mmol), 3-nitro-4-chlorophenyl isocyanate (0.264 mmol) and MP-carbonate (3.10 mmol/g, 0.792 mmol) in dichloromethane (3 mL) was shaken at room temperature overnight. The reaction mixture was eluted through a pre-wet (CH₂Cl₂) SCX-2 (2 g) cartridge with dichloromethane and evaporated to afford the desired product as a colourless gum (172.7 mg, 81%).

MS 541 [M+H]⁺

LC (80/20) 98.5%, 9.80 min

### Example 26

### 3-[2-(trifluoromethyl)-4′-chlorobenzhydryloxy]-N-(2,4-difluorophenyl)azetidine-1-carboxamide (47)

This material was prepared from 3-[2-(trifluoromethyl)-4′-chlorobenzhydryloxy)azetidine hydrochloride (15) and 2,4-difluorophenyl isocyanate using the procedure described for compound (46) (41 mg, 21%).

MS 497 [M+H]⁺

LC (80/20) 99.3%, 8.09 min

### Example 27

### 3-[2-(trifluoromethyl)-4′-chlorobenzhydryloxy]-N-(2,3-dihydrobenzo[1,4]dioxin-6-yl)azetidine-1-carboxamide (48)

This material was prepared from 3-[2-(trifluoromethyl)-4′-chlorobenzhydryloxy)azetidine hydrochloride (15) and 2,3-dihydrobenzo[1,4]dioxin-6-yl isocyanate using the procedure described for compound (46) (59 mg, 29%).

MS 519 [M+H]⁺

LC (80/20) 99.1%, 8.23 min

### Example 28

### 3-(2,4′-dichlorobenzhydryloxy)-N-(2,6-difluorophenyl)azetidine-1-carboxamide (49)

This material was prepared from 3-(2,4′-dichlorobenzhydryloxy)azetidine hydrochloride (4) (0.580 mmol) and 2,6-difluorophenyl isocyanate (0.580 mmol) using the procedure described for compound (46) (26.9 mg, 10%).

NMR (400 MHz, d⁶-DMSO) δ_{H} 2.53 (3H, s), 3.76 (1H, m), 3.85 (1H, m), 4.06 (1H, m), 4.12 (1H, m), 4.43 (1H, m), 5.82 (1H, s), 7.33-7.47 (8H, m), 7.53 (1H, m), 7.64 (1H, m), 7.79 (1H, m), 8.02 (1H, s), 8.64 (1H, s)

LC (80/20) 97.3%, 1.058 min

### Example 29

### 3-(4,4′-dichlorobenzhydryloxy)-N-(phenyl)azetidine-1-carboxamide (50)

To a stirred solution of 3-(4,4′-dichlorobenzhydryloxy)azetidine (34) (0.29 mmol) in dichloromethane (3 mL) was added phenyl isocyanate (0.290 mmol) and triethylamine (catalytic amount) and the reaction mixture was shaken at room temperature overnight. The reaction mixture was eluted through a pre-wet (CH₂Cl₂) SCX-2 (2 g) cartridge with dichloromethane then evaporated to afford the desired product as a white foam (72 mg, 58%).

NMR (400 MHz, d⁶-DMSO) δ_{H} 3.77 (2H, m), 4.05 (2H, m), 4.36 (1H, m), 5.60 (1H, s), 6.91 (1H, m), 7.21 (2H, m), 7.37-7.48 (10H, m), 8.44 (1H, s)

LC (50/80) 91.0%, 7.56 min

### Preparation of 2-chloro-N-methoxy-N-methylnicotinamide (51)

To a suspension of 2-chloronicotinic acid (78.8 mmol) in N,N-dimethylformamide (50 mL) was added N,N-carbonyldiimidazole (66.9 mmol) portionwise over 2 minutes. The reaction mixture was stirred at room temperature for 1.5 hours. N,O-dimethylhydroxylamine (118.2 mmol) was added and the reaction was stirred at room temperature for a further 6 hours. The mixture was diluted with water (200 mL) and extracted with ethyl acetate (3×75 mL). The organic extracts were washed with water (3×100 mL), brine (100 mL), dried (MgSO₄) and concentrated in vacuo to afford the desired product as a white solid (6.39 g, 40%).

NMR (400 MHz, CDCl₃) OH 3.30 (3H, br s), 3.56 (3H, br s), 7.30 (1H, m), 7.69 (1H, m), 8.46 (1H, s)

LC (50/80) 98.6%, 0.77 min

### Preparation of 4-chlorophenyl-(2-chloropyridin-3-yl)methanone (52)

To a stirred solution of 2-chloro-N-methoxy-N-methylnicotinamide (51) (20.5 mmol) in anhydrous tetrahydrofuran (30 mL) was added 4-chlorophenylmagnesium bromide (24.6 mmol) at ice bath temperature over 1 hour. The reaction was allowed to warm to room temperature and was stirred for 1 hour, then quenched with saturated ammonium chloride solution (500 mL) and extracted with diethyl ether (2×500 mL). The organic extracts were washed with water, brine, dried (MgSO₄) and concentrated in vacuo to afford the desired product an orange crystalline solid (5.80 g, 100%).

MS 253 [M+H]⁺

NMR (400 MHz, CDCl₃) δ_{H} 7.37-7.42 (1H, m), 7.42-7.49 (2H, m), 7.72-7.80 (3H, m), 8.57 (1H, m)

### Preparation of 4-chlorophenyl-(2-chloropyridin-3-yl)methanol (53)

To a stirred solution of 4-chlorophenyl-(2-chloropyridin-3-yl)methanone (52) (22.9 mmol) in ethanol (35 mL) was added sodium borohydride (I 1.5 mmol) portionwise at 0° C. The reaction mixture was allowed to warm to room temperature and was stirred for 1 hour. The reaction was quenched with water and the methanol was removed under reduced pressure. The aqueous residue was extracted with dichloromethane (400 mL) and the organic extracts were washed with water, brine, dried (MgSO₄) and concentrated in vacuo to yield a yellow oil. Purification by flash column chromatography [SiO₂; isohexane—ethyl acetate (8:1)→ethyl acetate] afforded the desired product as a yellow solid (4.32 g, 83%).

MS 255 [M+H]⁺

NMR (400 MHz, CDCl₃) δ_{H} 2.65 (1H, s), 6.13 (1H, s), 7.30 (5H, m), 7.97 (1H, m), 8.31 (1H, m)

### Preparation of 1-benzhydryl-3-[4-chloro-a-(2-chloropyridin-3-yl)benzyloxy]azetidine (54)

This compound was prepared from 4-chlorophenyl-(2-chloropyridin-3-yl)methanol (53) (16.8 mmol) and 1-benzhydryl-3-azetidinol (11.2 mmol) using the procedure described for 1-benzhydryl-3-(2,4′-dichlorobenzhydryloxy)azetidine (3). Purification by flash column chromatography [SiO₂; isohexane—ethyl acetate (2:1)] afforded the desired product as a colourless oil (0.88 g, 26%).

MS 476 [M+H]⁺

LC (50/80) 98.5%, 7.92 min

### Preparation of 3-[4-chloro-a-(2-chloropyridin-3-yl)benzyloxy]azetidine (55)

This compound was prepared from 1-benzhydryl-3-[4-chloro-□-(2-chloropyridin-3-yl)benzyloxy]azetidine (54) (1.85 mmol) rising the procedure described for 3-(2,4,4′-trichlorobenzhydryloxy)azetidine (10). Material was eluted through SCX-2 (2 g) with dichloromethane, then methanol, then 2N ammonia solution (in methanol). Evaporation afforded the desired product as a colourless oil (0.52 g, 91%).

MS 310[M+H]⁺

LC (50/80) 87.2%, 0.972 min

### Example 30

### 3-[4-chloro-□-(2-chloropyrid-3-yl)benzyloxy]-N-(1-adamantyl)azetidine-1-carboxamide (56)

A solution of 3-[4-chloro-□-(2-chloropyridin-3-yl)benzyloxy]azetidine (55) (0.38 mmol), 1-adamantyl isocyanate (0.38 mmol) and MP-carbonate (0.76 mmol) in anhydrous dichloromethane (3 mL) was shaken at room temperature overnight. The mixture was purified on a pre-packed silica cartridge eluting with isohexane:ethyl acetate (8:1→1:1) to afford the desired product as a white foam (101 mg, 55%).

MS 486 [M+H]⁺

LC (50/80) 95.3%, 7.21 min

### Preparation of 2-chlorophenyl-(2-chloropyridin-5-yl)methanol (57)

A solution of isopropylmagnesium chloride (2M in THF, 20 mmol) was added to a flame-dried flask charged with 2-chloro-5-iodopyridine (16.7 mmol) at −40° C. over 25 minutes. After a further 30 minutes, a solution of 2-chlorobenzaldheyde (20 mmol) in tetrahydrofuran (33 mL) was added in one portion. The reaction was allowed to warm to room temperature, brine (40 mL) was added and the resultant mixture was filtered through cellite. The phases were separated and the aqueous layer was extracted with diethyl ether (30 mL). The combined organic extracts were washed with brine (50 mL), dried (MgSO₄) and concentrated in vacuo to afford the desired product as a yellow oil 4.25 g, 100%).

MS 255 [M+H]⁺

LC (50/80) 98.0%, 2.66 min

### Preparation of 1-benzhydryl-3-[2-chloro-□-(2-chloropyridin-5-yl)benzyloxy]azetidine (58)

This material was prepared from 2-chlorophenyl-(2-chloropyridin-5-yl)methanol (57) (18.9 mmol) and 1-benzhydryl-3-azetidinol (1) using the procedure described for 1-benzhydryl-3-(2,4′-dichloroben:hydryloxy)azetidine (3). Purification by flash column chromatography [SiO₂; isohexane—ethyl aceate (4:1)] afforded the desired product as a colourless oil (0.85 g).

MS 476 [M+H]⁺

LC (50/80) 65%, 7.88 min

### Preparation of 3-[2-chloro-5-(2-chloropyridin-5-yl)benzyloxy]azetidine (59)

This compound was prepared from 1-benzhydryl-3-[2-chloro-□-(2-chloropyridin-5-yl)benzyloxy]azetidine (58) (1.77 mmol) using the procedure described for 3-(2,4,4′-trichlorobenzhydryloxy)azetidine (10). Material was eluted through SCX-2 (2 g) with dichloromethane, then methanol, then a 2N solution of ammonia (in methanol). Evaporation afforded the desired product as a colourless oil (0.35 g, 63%).

MS 310 [M+H]⁺

LC (50/80) 63.65, 1.04 min

### Example 31

### 3-[2-chloro-□-(2-chloropyridin-5-yl)benzyloxy]-N-(tert-butyl)azetidine-1-carboxamide (60)

A solution of 3-[2-chloro-□-(2-chloropyridin-5-yl)benzyloxy]azetidine (59) (0.38 mmol), tert-butyl isocyanate (0.40 mmol) and MP-carbonate (0.76 mmol) in anhydrous dichloromethane (3 mL) was shaken at room temperature overnight. The mixture was purified on a pre-packed silica cartridge eluting with isohexane—ethyl acetate (8:1-2:1) to afford the required product as a colourless gum (86.0 mg, 56%).

MS 408 [M+H]⁺

LC (50/80) 97.0%, 7.04 min

### Example 32

### 3-[4-chloro-□-(2-chloropyridin-3-yl)benzyloxy]-N-(tert-butyl)azetidine-1-carboxamide (61)

A mixture of 3-[4-chloro-□-(2-chloropyridin-3-yl)benzyloxy]azetidine (55) (0.43 mmol), tert-butyl isocyanate (0.43 mmol) and MP-carbonate (0.76 mmol) in anhydrous dichloromethane (3 mL) was shaken at room temperature overnight. The mixture was purified on a pre-packed silica cartridge eluting with isohexane:ethyl acetate (8:1→1:1) to afford the desired product as a white foam (88 mg, 50%).

MS 408 [M+H]⁺

LC (50/80) 95.3%, 7.21 min

### Example 33

### 3-[(R*)-4-chloro-□-(2-chloropyridin-3-yl)benzyloxy]-N-(tert-butyl)azetidine-1-carboxamide (62); and

### Example 34

### 3-[(S*)-4-chloro-□-(2-chloropyridin-3-yl)benzyloxy]-N-(tert-butyl)azetidine-1-carboxamide (63)

The racemic compound 3-[4-chloro-□-(2-chloropyridin-3-yl)benzyloxy]-N-(tert-butyl)azetidine-1-carboxamide (61) was separated into samples which were significantly enriched in each single enantiomeric form. Thus, a sample of the racemic mixture (100 mg) was dissolved in 1 mL of an IPA—hexane (10:90) mix, plus DCM (100 □L) to fully dissolve. This mixture was repeatedly injected (6×50 □L) onto a Daicel Chiralpak® AD™ chiral HPLC column (250 mm×21 mm ID) fitted with a guard column (50 mm×21 mm ID) [eluent: IPA—hexane (10:90); flow-rate: 10 mL/min; wavelength: 235 nm]. The combined first-eluting enantiomer (arbritrarily assigned R* unknown absolute stereochemistry) fractions were evaporated to afford 3-[(R*)-4-chloro-□-(2-chloropyrid-3-yl)benzyloxy]-N-(tert-butyl)azetidine-1-carboxamide (10.4 mg, 38.1%).

MS 408 [M+H]⁺

LC (50/80) 98.4%, 4.84 min

LC [Chiral AD; IPA—hexane (10:90)] 90.0%, 19.97 min; 100% ee

The combined second-eluting enantiomer (arbritrarily assigned S* unknown absolute stereochemistry) fractions were evaporated to afford 3-[(S*)-4-chloro-□-(2-chloropyrid-3-yl)benzyloxy]-N-(tert-butyl)azetidine-1-carboxamide (15.0 mg, 56.8%).

MS 408 [M+H]⁺

LC (50/80) 88.7%, 4.86 min

LC [Chiral AD; IPA—hexane (10:90)] 72.8%, 29.43 min; 16.1%, 20.09 min; 64% ee

### Example 35

### 3-[2-(trifluoromethyl)-4′-ehlorobenzhydryloxy]-N-(benzoyl)azetidine-1-carboxamide (64)

This material was prepared from 3-[2-(trifluoromethyl)-4′-chlorobenzhydryloxy]azetidine hydrochloride (15) and methyl (S)-(−)-2-isocyanato-3-phenylpropionate using the procedure described for 3-(4,4′-dichlorobenzhydryloxy)-N-(phenyl)azetidine-1-carboxamide (50) (90.9 mg, 63%).

NMR (400 MHz, d⁶-DMSO) δ_{H} 2.86 (1H, m), 2.96 (1H, m), 3.48 (1H, m), 3.63 (1H, m), 3.76-3.92 (2H, m), 4.20-4.31 (2H, m), 2.73 (1H, s), 6.75 (1H, m), 7.15-7.32 (7H, m), 7.43 (2H, m), 7.57 (1H, m), 7.74 (3H, m)

LC (50/80) 96.0%, 7.40 min

### Preparation of 2-(trifluoromethyl)-4′-fluorobenzhydrol (65)

This material was prepared from 2-(trifluoromethyl)phenylmagnesium bromide (16 mmol) and 4-fluorobenzaldehyde (1.64 mL, 15 mmol) using the procedure described for 2-(trifluoromethyl)-4′-chlorobenzhydrol (13) (4.27 g, 100%).

LC (50/80) 99.3%, 4.37 min

### Preparation of 1-benzhydryl-3-[2-(trifluoromethyl)-4′-fluorobenzhydryloxy]azetidine (66)

This material was prepared from 1-benzhydryl-3-azetidinol (1) (7.5 mmol) and 2-(trifluoromethyl)-4′-fluorobenzhydrol (72) (15 mmol) using the procedure described for 1-benzhydryl-3-[2-(trifluoromethyl)-4′-chlorobenzhydryloxy]azetidine (14). After basic aqueous workup, the crude product was used in the next step without further purification.

### Preparation of 3-[2-(trifluoromethyl)-4′-fluorobenzhydryloxy]azetidine hydrochloride (67)

This material was prepared from 1-benzhydryl-3-[2-(trifluoromethyl)-4′-fluorobenzyloxy]azetidine (66) (7.5 mmol) using the procedure described for 3-[2-(trifluoromethyl)-4′-chlorobenzhydryloxy]azetidine hydrochloride (15). Crystallisation from DIPE-MeOH afforded the product as a white solid (1.49 g, 55%).

LC (50/80) 99.6%, 2.30 min

### Example 36

### 3-[2-(trifluoromethyl)-4′-fluorobenzhydryloxy]-N-(benzoyl)azetidine-1-carboxamide (68)

A mixture of 3-[2-(trifluoromethyl)-4′-fluorobenzhydryloxy]azetidine hydrochloride (67) (150 mg, 0.42 mmol), MP-carbonate (2.62 mmol/g; 475 mg, 1.24 mmol) and anhydrous DCM (4 mL) was shaken at ambient temperature for 5 min. Molecular sieves were added, followed by benzoyl isocyanate (tech. Grade; 68 mg, 0.42 mmol). The mixture was shaken for 16 h, then poured onto a DCM-wetted SCX-2 (1 g). Elution with DCM (24 mL) and evaporation afforded the desired product as a colourless foam which gave a white solid on scratching (155 mg, 79%).

MS 473 [M+H]⁺

LC (50/80) 86.3%, 6.51 min

### Example 37

### 3-(2,4′-dichlorobenzhydryloxy)-N-(piperidyl)azetidine-1-carboxamide (69)

To a stirred solution of triphosgene (0.11 mmol) in anhydrous dichloromethane (10 mL) at ice bath temperature was added a solution of 3-(2,4′-dichlorobenzhydryloxy)azetidine hydrochloride (4) (0.25 mmol) and pyridine (0.55 mmol) in anhydrous dichloromethane (15 mL). The reaction mixture was stirred at room temperature for 4 hours, then filtered through a silica pad and reduced in vacuo to give a yellow oil. This material was dissolved in anhydrous dichloromethane (5 mL), triethylamine (0.55 mmol), and 1-aminopiperidine (0.55 mmol) were added and the reaction was shaken at room temperature overnight. The reaction mixture was eluted through a pre-packed silica cartridge (isohexane:ethylacetate-1:1) to give a yellow oil. This material was dissolved in diethyl ether (5 mL) and HCl (4M in 1,4-dioxane, 0.4 mL) was added. After 1 hour of stirring at room temperature, the resultant white solid was isolated by filtration (16.0 mg, 15%).

MS 434 [M+1]⁺

LC (50/80) 96.2%, 6.95 min

### Preparation of 3-(4,4′-dichlorobenzhydryloxy)azetidine-1-carbonyl chloride (70)

3-(4,4′-dichlorobenzhydryloxy)azetidine hydrochloride (34) was converted to the corresponding free-base using standard methods.

To a stirred solution of triphosgene (1.74 mmol) in anhydrous dichloromethane (30 mL) at ice bath temperature was added a solution of 3-(4,4′-dichlorobenzhydryloxy)azetidine (4.58 mmol) and pyridine (5.04 mmol) in anhydrous dichloromethane (15 mL). The reaction mixture was stirred at room temperature for 4 hours, then filtered through a silica pad and reduced in vacuo to afford the desired product as a yellow oil (1.33 g, 78%).

NMR (400 MHz, d⁶-DMSO) δ_{H} 3.90 (1H, m), 4.04 (1H, m), 4.16 (1H, m), 4.30 (2H, m), 5.63 (1H, s), 7.35-7.45 (8H, m)

### Example 38

### 3-(4,4′-dichlorobenzhydryloxy)-N-(piperidyl)azetidine-1-carboxamide (71)

A solution of 3-(4,4′-dichlorobenzhydryloxy)azetidine-1-carbonyl chloride (70) (0.269 mmol), 1-aminopiperidine (0.540 mmol), and triethylamine (0.540 mmol) hi anhydrous dichloromethane (3 mL) was shaken at room temperature overnight. The reaction mixture was eluted through a pre-wet (CH₂Cl₂) SCX-2 (2 g) cartridge with dichloromethane then evaporated to afford the desired product as a colourless gum (31 mg, 24%).

NMR (400 MHz, d⁶-DMSO) δ_{H} 1.25 (2H, br s), 1.46 (4H, br m), 2.56 (4H, br m), 3.65 (2H, m), 3.92 (2H, m), 4.29 (1H, m), 5.56 (1H, s), 7.27 (1H, s), 7.36-7.45 (8H, m)

LC (50/80) 99.8%, 7.41 min

### Example 39

### 3-(2,4′-dichlorobenzhydryloxy)-N-(phenylsulfonyl)azetidine-1-carboxamide (72)

To a solution of 3-(2,4′-dichlorobenzhydryloxy)azetidine hydrochloride (4) (0.58 mmol) in dichloromethane (3 mL) was added benzene sulphonyl isocyanate (0.58 mmol) and MP-carbonate (3.01 mmol/g, 1.74 mmol) and the reaction mixture was shaken at room temperature overnight. The reaction mixture was eluted through a pre-wet (CH₂Cl₂) SCX-2 (2 g) cartridge with dichloromethane to yield the desired product as a colourless gum (50.0 mg, 18%).

NMR (400 MHz, d⁶-DMSO) δ_{H} 3.62-3.80 (2H, br m), 3.90-4.08 (2H, br m), 4.34 (1H, m), 5.75 (1H, s), 7.32-7.45 (7H, m), 7.55-7.68 (4H, m), 7.87 (2H, m), 10.9 (1H, br s)

LC (50/80) 96.6%, 4.10 min

### Preparation of (R)-1-benzyl-3-(4,4′-dichlorobenzhydryloxy)pyrrolidine (73)

A solution of (R)-(+)-1-benzyl-3-pyrrolidinol (55 mmol), 4,4′-dichlorobenzhydrol (113 mmol) and p-toluenesulphonic acid (113 mmol) in toluene (150 mL) was heated to reflux in a Dean-Stark apparatus overnight. The solution was cooled, washed with 10% sodium hydroxide solution, dried (MgSO₄) and concentrated in vacuo. The residue was purified by flash column chromatography [SiO₂; isohexane→ethyl acetate-isohexane (1:4)] to furnish the product as a brown oil (15.1 g, 67%).

NMR (400 MHz, d⁶-DMSO) δ_{H} 1.75 (1H, m), 2.00 (1H, m), 2.38 (1H, m), 2.53 (1H, m), 2.60 (2H, m), 3.55 (2H, m), 4.00 (1H, m), 5.51 (1H, s), 7.21-7.42 (13H, m)

LC (50/80) 98.7%, 8.20 min

### Preparation of (R)-3-(4,4′-dichlorobenzhydryloxy)pyrrolidine (74)

To a stirred solution of (R)-1-benzyl-3-(4,4′-dichlorobenzhydryloxy)pyrrolidine (73) (12 mmol) in 1,2-dichloroethane (50 mL) was added 1-chloroethyl chloroformate (24 mmol) dropwise at 0° C. and the reaction mixture stirred at reflux overnight. The reaction mixture was cooled to room temperature and concentrated in vacuo, then dissolved in methanol (50 mL) and stirred at reflux overnight. The reaction mixture was concentrated in vacuo, then diluted with ethyl acetate (400 mL) and washed with sodium hydroxide (5N, 400 mL), dried (MgSO₄) and concentrated in vacuo to furnish a yellow oil. Purification by flash column chromatography [SiO₂; ethyl acetate-methanol-ammonium hydroxide (90:8:2)] afforded the desired product as a brown viscous oil (1.79 g, 46%).

NMR (400 MHz, d⁶-DMSO) δ_{H} 1.72 (2H, m), 2.64-2.90 (4H, m), 3.94 (1H, m), 5.55 (1H, s), 7.33-7.44 (8H, m)

LC (50/80) 96.8%, 3.58 min

### Example 40

### (R)-3-(4,4′-dichlorobenzhydryloxy)-N-(allyl)pyrrolidine-1-carboxamide (75)

A solution of (R)-3-(4,4′-dichlorobenzhydryloxy)pyrrolidine (74) (0.31 mmol), allyl isocyanate (0.31 mmol) and triethylamine (catalytic) in anhydrous dichloromethane was shaken at room temperature overnight. The reaction mixture was eluted through a pre-wet (CH₂Cl₂) SCX-2 (2 g) cartridge with dichloromethane and evaporated to afford the required product as a colourless gum (93.2 mg, 74%).

NMR (400 MHz, d⁶-DMSO) δ_{H} 1.89 (1H, m), 2.00 (1H, m), 3.31 (4H, m), 3.63 (2H, m), 4.04 (1H, m), 4.99 (1H, m), 5.07 (1H, m), 5.64 (1H, s), 5.79 (1H, m), 6.28 (1H, m), 7.33-7.43 (8H, m)

LC (50/80) 95.8%, 6.51 min

### Example 41

### (R)-3-(4,4′-dichlorobenzhydryloxy)-N-(tert-butyl)pyrrolidine-1-carboxamide (76)

This material was formed from (R)-3-(4,4′-dichlorobenzhydryloxy)pyrrolidine (74) and tert-butyl isocyanate, using the procedure described for (R)-3-(4,4′-dichlorobenzhydryloxy)-N-(allyl)pyrrolidine-1-carboxamide (75) (100 mg, 77%).

NMR (400 MHz, d⁶-DMSO) δ_{H} 1.25 (9H, s), 1.86 (1H, m) 1.99 (1H, m), 3.30 (4H, m), 4.04 (1H, m), 5.19 (1H, s), 5.65 (1H, s), 7.34-7.42 (8H, m)

LC (50/80) 99.0%, 7.16 min

### Example 42

### (R)-3-(4,4′-dichlorobenzhydryloxy)-N-(benzyl)pyrrolidine-1-carboxamide (77)

This material was formed from (R)-3-(4,4′-dichlorobenzhydryloxy)pyrrolidine (74) and benzyl isocyanate, using the procedure described for (R)-3-(4,4′-dichlorobenzhydryloxy)-N-(allyl)pyrrolidine-1-carboxamide (75) (118 mg, 84%).

NMR (400 MHz, d⁶-DMSO) δ_{H} 1.92 (1H, m), 2.02 (1H, m), 3.34 (4H, m), 4.04 (1H, m), 4.23 (2H, m), 5.65 (1H, s) 6.70 (1H, m), 7.18-7.44 (13H, m)

LC (50/80) 98.3%, 7.29 min

### Preparation of (R)-3-(4,4′-dichlorobenzhydryloxy)pyrrolidine-1-carbonyl Chloride (78)

To a stirred solution of triphosgene (2.91 mmol), in dichloromethane (25 mL) at ice bath temperature was added dropwise a solution of (R)-3-(4,4′-dichlorobenzhydryloxy)pyrrolidine (74) (7.66 mmol) and pyridine (8.43 mmol) in dichloromethane (25 mL). The mixture was stirred at ambient temperature for 3 hours, filtered through a silica pad, eluting with dichloromethane, and evaporated. Trituration with diisopropyl ether: isohexane furnished the desired product as a white solid (2.06 g, 70%).

NMR (400 MHz, d⁶-DMSO) δ_{H} 1.92-2.16 (2H, m), 3.39-3.71 (4H, m), 4.12 (1H, m), 5.71 (1H, s), 7.35-7.43 (8H, m)

### Example 43

### (R)-1-(1-piperidinecarbonyl)-3-(4,4′-dichlorobenzhydryloxy)pyrrolidine (79)

A solution of (R)-3-(4,4′-dichlorobenzhydryloxy)pyrrolidine-1-carbonyl chloride (78) (0.260 mmol), piperidine (0.860 mmol) and triethylamine (0.520 mmol) in anhydrous dichloromethane (3 mL) was shaken at room temperature overnight. The reaction mixture was eluted through a pre-wet (CH₂Cl₂) SCX-2 (2 g) cartridge with dichloromethane to afford the desired product as a colourless gum (52 mg, 46%).

NMR (400 MHz, d⁶-DMSO) δ_{H} 1.34-1.55 (6H, m), 1.84 (1H, m), 1.96 (1H, m), 2.98-3.13 (4H, m), 3.23 (2H, m), 3.43 (2H, m), 4.04 (1H, m), 5.65 (1H, s), 7.31-7.44 (8H, m)

LC (50/80) 99.6%, 7.58 min

### Example 44

### (R)-1-(1-pyrrolidinecarbonyl)-3-(4,4′-dichlorobenzhydryloxy)pyrrolidine (80)

This material was formed from (R)-3-(4,4′-dichlorobenzhydryloxy)pyrrolidine-1-carbonyl chloride (78) and pyrrolidine, using the procedure described for (R)-1-(piperidine-1-carbonyl)-3-(4,4′-dichlorobenzhydryloxy)pyrrolidine (79) (13.0 mg, 12%).

NMR (400 MHz, d⁶-DMSO) δ_{H} 1.64-2.00 (6H, m), 3.15-3.45 (8H, m), 4.03 (1H, m), 5.66 (1H, s), 7.34-7.44 (8H, m)

LC (50/80) 99.8%, 6.92 min

### Preparation of (S)-1-benzyl-3-(4,4′-dichlorobenzhydryloxy)pyrrolidine (81)

A solution of (S)-(−)-1-benzyl-3-pyrrolidinol (56 mmol), 4,4′-dichlorobenzhydrol (113 mmol) and p-toluenesulphonic acid (113 mmol) in toluene (150 mL) was heated to reflux in a Dean-Stark apparatus overnight. The solution was cooled, washed with 10% sodium hydroxide solution, dried (MgSO₄) and concentrated in vacuo. The residue was purified by flash column chromatography [SiO₂; isohexane→diisopropyl ether] to furnish the product as a brown oil (6.448 g, 28%).

NMR (400 MHz, d⁶-DMSO) δ_{H} 1.75 (1H, m), 1.98 (1H, m), 2.38 (1H, m), 2.53 (1H, m), 2.60 (2H, m), 3.55 (2H, m), 4.00 (1H, m), 5.51 (1H, s), 7.21-7.42 (13H, m)

LC (50/80) 95.4%, 8.16 min

### Preparation of (S)-3-(4,4′-dichlorobenzhydryloxy)pyrrolidine (82)

To a stirred solution of (S)-1-benzyl-3-(4,4′-dichlorobenzhydryloxy)pyrrolidine (81) (12 mmol) in 1,2-dichloroethane (50 mL) was added 1-chloroethyl chloroformate (24 mmol) dropwise at 0° C. and the reaction mixture stirred at reflux overnight. The reaction mixture was cooled to room temperature and concentrated in vacuo, then dissolved in methanol (50 mL) and stirred at reflux overnight. The reaction mixture was concentrated in vacuo, then diluted with ethyl acetate (400 mL) and washed with sodium hydroxide (5N, 400 mL), dried (MgSO₄) and concentrated in vacuo to furnish a yellow oil. The residue was purified by flash chromatography [SiO₂; ethyl acetate-methanol-ammonium hydroxide (90:8:2)] to afford the desired product as a brown viscous oil (1.78 g, 45%).

NMR (400 MHz, d⁶-DMSO) δ_{H} 1.72 (2H, m), 2.64-2.90 (4H, m), 3.94 (1H, m), 5.55 (1H, s), 7.33-7.44 (8H, m)

LC (50/80) 99.2%, 3.32 min

### Example 45

### (S)-3-(4,4′-dichlorobenzhydryloxy)-N-(cyclohexyl)pyrrolidine-1-carboxamide (83)

A solution of (S)-3-(4,4′-dichlorobenzhydryloxy)pyrrolidine (82) (0.31 mmol), cyclohexyl isocyanate (0.31 mmol) and triethylamine (catalytic) in anhydrous dichloromethane was shaken at room temperature overnight. The reaction mixture was eluted through a pre-wet (CH₂Cl₂) SCX-2 (2 g) cartridge with dichloromethane and evaporated to afford the desired product as a colourless gum (112 mg, 81%).

NMR (400 MHz, d⁶-DMSO) δ_{H} 0.99-1.28 (5H, m), 1.51-1.76 (5H, m), 1.86 (1H, m), 2.00 (1H, m), 3.19-3.42 (4H, m), 4.03 (1H, m), 5.62 (1H, s), 5.68 (1H, m), 7.35-7.42 (8H, m)

LC (50/80) 98.4%, 7.56 min

### Example 46

### (S)-3-(4,4′-dichlorobenzhydryloxy)-N-(benzyl)pyrrolidine-1-carboxamide (84)

This material was formed from (S)-3-(4,4′-dichlorobenzhydryloxy)pyrrolidine (82) and benzyl isocyanate, using the procedure described for (S)-3-(4,4′-dichlorobenzhydryloxy)-N-(cyclohexyl)pyrrolidine-1-carboxamide (83) (120 mg, 85%).

NMR (400 MHz, d⁶-DMSO) δ_{H} 1.92 (1H, m), 2.03 (1H, m), 3.24-3.47 (4H, m), 4.06 (1H, m), 4.22 (2H, d, J=6.0 Hz), 5.65 (1H, s), 6.70 1H, m), 7.17-7.44 (13H, m)

LC (50/80) 98.6%, 7.28 min

### Example 47

### (S)-3-(4,4′-dichlorobenzhydryloxy)-N-(1-adamantyl)pyrrolidine-1-carboxamide (85)

This material was formed from (S)-3-(4,4′-dichlorobenzhydryloxy)pyrrolidine (82) and 1-adamantyl isocyanate, using the procedure described for (S)-3-(4,4′-dichlorobenzhydryloxy)-N-(cyclohexyl)pyrrolidine-1-carboxamide (83) (133 mg, 86%).

NMR (400 MHz, d⁶-DMSO) δ_{H} 1.59 (6H, m), 1.81-2.01 (13H, m), 3.20-3.40 (9H, m), 4.02 (2H, m), 5.04 (1H, s), 5.64 (1H, s), 7.34-7.43 (8H, m)

LC (50/80) 99.5%, 8.86 min

### Example 48

### 1-benzyl-4-(2,4′-dichlorobenzhydryloxy)piperidine (86)

To a solution of N-benzyl-4-piperidinol (10.7 mmol) in toluene (100 ml) was added 2,4′-dichlorobenzhydrol (2) (21.3 mmol) and para-toluenesulfonic acid (21.3 mmol). The reaction mixture was heated to reflux for 1 hr, cooled and partitioned between ethyl acetate and aq. sodium bicarbonate. The organic phase was separated and washed with water and brine, dried (MgSO₄) and concentrated under reduced pressure to afford the crude product, which was diluted with iso-hexane-ethyl acetate (5:1). After standing for 1 hA, the white solid that had precipitated was removed by filtration (bis-ether by-product) and the liquors concentrated under reduced pressure to afford the title compound as a colourless oil (3.71 g, 81%).

MS 426 [M+H]⁺

LC (50/80) 99.0%, 8.02 min

### Preparation of 4-(2,4′-dichlorobenzhydryloxy)piperidine (87)

To a solution of 1-benzyl-4-(2,4′-dichlorobenzhydryloxy)piperidine (86) (3.22 mmol) in dichloromethane (30 ml) at 0° C. was added 1-chloroethyl chloroformate (7.08 mmol). The reaction mixture was stirred for 15 mins and then warmed to ambient temperature and stirred for 3 hrs. The solvent was evaporated and the residue diluted with methanol and stirred for a further 2 hrs at ambient temperature and then at reflux for 45 mins. The cooled solution was washed with sodium hydroxide (1.0 M, water, dried (MgSO₄) and concentrated under reduced pressure to afford the title compound as a pale yellow oil (0.78 g, 65%).

MS 336 [M+H]⁺

LC (50/80) 99.2%, 4.14 min

### Example 49

### 4-(2,4′-dichlorobenzhydryloxy)-N-(1-adamantyl)piperidin-1-carboxamide (88)

To a solution of 4-(2,4′-dichlorobenzhydryloxy)piperidine (87) (0.30 mmol) in dichloromethane (10 ml) was added 1-adamantyl isocyanate (0.30 mmol) and triethylamine (0.45 mmol). The reaction mixture was shaken for 18 hrs, trisamine resin (0.45 mmol) added and the solution shaken for a further 1 hr before the reaction mixture was eluted through a pre-wetted (dichloromethane) SCX-2 (2 g) cartridge with dichloromethane to yield the title compound as a white solid (135 mg, 88%).

MS 513 [M+R]⁺

LC (50/80) 97.8%, 8.99 min

### Preparation of 4-(2,4′-dichlorobenzhydryloxy)piperidine-1-carbonyl Chloride (89)

To a solution of 1-benzyl-4-(2,4′-dichlorobenzhydryloxy)piperidine (86) (4.06 mmol) in dichloromethane (50 ml) at ambient temperature was added phosgene (4.90 mmol, 1.75 M solution in toluene) and the solution stirred for 1 hr after which time the solvent was evaporated, under reduced pressure, to afford the title compound as a yellow oil which was used immediately, without further purification.

### Example 50

### 1-(1-piperidinecarbonyl)-4-(2,4′-dichlorobenzhydryloxy)piperidine (90)

To a solution of 4-(2,4′-dichlorobenzhydryloxy)piperidine-1-carbonyl chloride (89) (4.06 mmol) in dichloromethane (40 ml) was added piperidine (4.06 mmol) and triethylamine (8.12 mmol). The reaction mixture was stirred for 18 hrs then partitioned between water and ethyl acetate. The organic phase was separated, dried (MgSO₄) and purified by chromatography on silica gel (eluant 20% ethyl acetate in iso-hexane) before elution through a pre-wetted (dichloromethane) SCX-2 (2 g) cartridge with dichloromethane to yield the title compound as a colourless oil (1.22 g, 67%).

MS 447 [M+H]⁺

LC (50/80) 99.3%, 7.88 min

### Preparation of 1-benzyl-4-(2-methylbenzhydryloxy)piperidine (91)

This material was prepared from N-benzyl-4-piperidinol (20.9 mmol) and 2-methylbenzhydrol (41.8 mmol) using the method described for 1-benzyl-4-(2,4′-dichlorobenzhydryloxy)piperidine (86). Purification by chromatography (iso-hexane/ethyl acetate, 3:1) afforded the title compound as a pale yellow oil (6.5 g, 83%)

MS 372 [M+H]⁺

LC (50/80) 89.7%, 6.51 min

### Preparation of 4-(2-methylbenzhydryloxy)piperidine (92)

This material was prepared from 1-benzyl-4-(2-methylbenzhydryloxy)piperidine (91) (3.81 mmol) using the method described for 4-(2,4′-dichlorobenzhydryloxy)piperidine (87). The material was used directly without purification.

### Example 51

### 1-benzyl-4-(2,4,4′-trichlorobenzhydryloxy)piperidine (93)

This material was prepared from N-benzyl-4-piperidinol and 2,4,4′-trichlorobenzhydrol (8) using the method describe for 1-benzyl-4-(2,4′-dichlorobenzhydryloxy)piperidine (86) (0.83 g, 81%).

MS 460 [M+H]⁺

LC (50/80) 99.1%, 9.55 min

### Preparation of 4-(2,4,4′-trichlorobenzhydryloxy)piperidine (94)

To a solution of 1-benzyl-4-(2,4,4′-trichlorobenzhydryloxy)piperidine (93) (2.17 mmol) in 1,2-dichloroethane (30 ml) at ambient temperature was added 1-chloroethyl chloroformate (4.78 mmol). The reaction mixture was heated to reflux for 4 hrs and then cooled to ambient temperature. The solvent was evaporated and the residue diluted with methanol and stirred for a further 2 hrs at reflux. The cooled solution was eluted through a pre-wetted (dichloromethane) SCX-2 (2 g) cartridge with dichloromethane followed by ammonia (2.0 M in methanol) to yield the title compound as a colourless oil (473 mg, 59%).

MS 370 [M+H]⁺

LC (50/80) 99.8%, 5.83 min

### Example 52

### 4-(2,4,4′-trichlorobenzhydryloxy)-N-(tert-butyl)piperidine-1-carboxamide (95)

To a stirred solution of 4-(2,4,4′-trichlorobenzhydryloxy)piperidine (94) (0.27 mmol) in dichloromethane (10 mL) was added tert-butyl isocyanate (0.27 mmol) and triethylamine (0.41 mmol) and the reaction mixture was stirred at ambient temperature for 17 hours. The reaction mixture was eluted through a pre-wetted (dichloromethane) SCX-2 (2 g) cartridge with dichloromethane and evaporated to yield the title compound as a white solid (92 mg, 62%).

MS 469 [M+H]⁺

LC (50/80) 99.8%, 8.14 min

### Example 53

### 4-(2,4,4′-trichlorobenzhydryloxy)-N-(cyclohexyl)piperidine-1-carboxamide (96)

This material was prepared from 4-(2,4,4′-trichlorobenzhydryloxy)piperidine (94) and the corresponding commercially available isocyanate, using the procedure described for compound (88) (117 mg, 87%).

MS 495 [M+H]⁺

LC (50/80) 99.7%, 8.71 min

### Preparation of 4-(2,4,4′-trichlorobenzhydryloxy)piperidine-1-carbonyl chloride (97)

To a solution of 1-benzyl-4-(2,4,4′-trichlorobenzhydryloxy)piperidine (93) (1.65 mmol) in dichloromethane (20 ml) at ambient temperature was added phosgene (1.98 mmol, 1.75 M solution in toluene) and the solution stirred for 2 hr after which time the solvent was removed, under reduced pressure, to afford the title compound as a yellow oil that was used immediately, without further purification.

### Example 54

### 4-(2,4,4′-trichlorobenzhydryloxy)-N-methyl-N-benzylpiperidine-1-carboxamide (98)

To a solution of 4-(2,4,4′-trichlorobenzhydryloxy)piperidine-1-carbonyl chloride (97) (0.50 mmol) in dichloromethane (10 ml) was added N-methylbenzylamine (0.55 mmol). The solution was stirred at ambient temperature for 18 hr, and then eluted through a pre-wetted (dichloromethane) SCX-2 (2 g) cartridge with dichloromethane-methanol (1:1) and evaporated to yield the title compound as a colourless oil (195 mg, 75%).

MS 517 [M+H]⁺

LC (50/80) 97.1%, 9.24 min

### Example 55

### 1-(1-piperidinecarbonyl)-4-(2,4,4′-trichlorobenzhydryloxy)piperidine (99)

To a solution of 4-(2,4,4′-trichlorobenzhydryloxy)piperidine-1-carbonyl chloride (97) (0.50 mmol) in dichloromethane (10 ml) was added piperidine (0.55 mmol). The solution was stirred at ambient temperature for 18 hr, and then eluted through a pre-wetted (dichloromethane) SCX-2 (2 g) cartridge with dichloromethane-methanol (1:1) and evaporated to yield the title compound as a colourless oil (163 mg, 68%).

MS 483 [M+H]⁺

LC (50/80) 97.3%, 8.67 min

### Example 56

### 4-(2,4′-dichlorobenzhydryloxy)-N-(tert-butyl)piperidine-1-thiocarboxamide (100)

To a solution of 4-(2,4′-dichlorobenzhydryloxy)piperidine (87) (0.30 mmol) in dichloromethane (10 ml) was added tert-butyl isothiocyanate (0.30 mmol) and MP-carbonate resin (0.90 mmol). The reaction mixture was shaken for 18 hrs, trisamine resin (0.45 mmol) added and the solution shaken for a further 1 hr before the reaction mixture was eluted through a pre-wetted (dichloromethane) SCX-2 (2 g) cartridge with dichloromethane to yield the title compound as a white solid (66 mg, 49%).

MS 451 [M+H]⁺

LC (50/80) 93.2%, 7.99 min

### Example 57

### 1-(tert-butylacetyl)-4-(2,4′-dichlorobenzhydryloxy)piperidine (101)

To a solution of 4-(2,4′-dichlorobenzhydryloxy)piperidine (87) (0.80 mmol) in dichloromethane (10 ml) was added MP-carbonate (2.41 mmol) and tert-butylacetyl chloride (0.80 mmol). The solution was stirred for 18 hr at ambient temperature, and then eluted through a pre-wetted (dichloromethane) SCX-2 (2 g) cartridge with dichloromethane-methanol (1:1) to yield the title compound as a colourless oil (294 mg, 84%).

MS 434 [M+H]⁺

LC (50/80) 97.1%, 7.71 min

### Example 58

### 1-(tert-butylacetyl)-4-(2-methylbenzhydryloxy)piperidine (102)

This material was prepared from 4-(2-methylbenzhydryloxy)piperidine (92) and tert-butylacetylchloride, using the procedure described for 1-(tert-butylacetyl)-4-(2,4′-dichlorobenzhydryloxy)piperidine (101) (38 mg 12%).

MS 380 [M+H]⁺

LC (50/80) 81.0%, 6.72 min

### Preparation of 1-benzyl-4-(4,4′-dichlorobenzhydryloxy)piperidine (103)

This material was prepared from N-benzyl-4-piperidinol (5.23 mmol) and 4,4′-dichlorobenzhydrol (15.7 mmol) using the method described for 1-benzyl-4-(2,4′-dichlorobenzhydryloxy)piperidine (86). Purification by chromatography (iso-hexane then 20% ethyl acetate/iso-hexane) afforded the title compound as a pale yellow oil (1.19 g, 53%).

NMR (400 MHz, CDCl₃) δ_{H} 1.69 (2H, m), 1.82 (2H, m), 2.15 (2H, m), 2.77 (2H, m), 3.40 (1H, m), 3.48 (2H, s), 5.42 (1H, s), 7.20-7.33 (13H+CHCl₃, m)

LC (50/80) 99.3%, 8.50 min

### Preparation of 4-(4,4′-dichlorobenzhydryloxy)piperidine (104)

This material was prepared from 1-benzyl-4-(4,4′-dichlorobenzhydryloxy)piperidine

(103) (3.81 mmol) using the method described for the preparation of 4-(2,4′-dichlorobenzhydryloxy)piperidine (87). Trituration of the initial product with diethyl ether afforded the title compound as a white solid (1.86 g, 93%).

NMR (400 MHz, d₆ DMSO) δ_{H} 1.75 (2H, m), 1.98 (2H, m), 2.90 (2H, m), 3.18 (2H, m), 3.60 (1H, m), 5.77 (1H, s), 7.38-7.43 (5H, m), 8.38-8.50 (2H, br).

LC (50/80) 99.6%, 4.58 min

### Example 59

### 1-(4-fluorophenylsulfonyl)-4-(4,4′-dichlorobenzhydryloxy)piperidine (105)

A mixture of 4-(4,4′-dichlorobenzhydryloxy)piperidine (104), 4-fluorophenylsulfonyl chloride and MP-carbonate (3.10 mmol/g) in anhydrous dichloromethane (3 mL) was shaken at room temperature overnight. AP-trisamine was added and the mixture was shaken at room temperature for a further 2 hours, filtered through PTFE cartridges and reduced in vacuo to afford the required product as a colourless gum (13.0 mg).

MS 494 [M+H]⁺

LC (50/80) 96.8%, 8.23 min

### Example 60

### 1-(2-fluorophenylsulfonyl)-4-(4,4′-dichlorobenzhydryloxy)piperidine (106)

This compound was prepared from 4-(4,4′-dichlorobenzhydryloxy)piperidine (104) and 2-fluorophenylsulfonyl chloride using the procedure described for 1-(4-fluorophenylsulfonyl)-4-(4,4′-dichlorobenzhydryloxy)piperidine (105).

(10.3 mg)

MS 494 [M+H]⁺

LC (50/80) 96.3%, 8.10 min

### Example 61

### 1-(3-chloropropanesulfonyl)-4-(4,4′-dichlorobenzhydryloxy)piperidine (107)

This compound was prepared from 4-(4,4′-dichlorobenzhydryloxy)piperidine (104) and 3-chloropropanesulphonyl chloride using the procedure described for 1-(4-fluorophenylsulfonyl)-4-(4,4′-dichlorobenzhydryloxy)piperidine (105). (6.1 mg)

MS 476 [M+H]⁺

LC (50/80) 91.8%, 7.70 min

### Example 62

### 4-(2,4′-dichlorobenzhydryloxy)-O-(tert-butyl)piperidine-1-carboxylate (108)

To a stirred solution of 4-(2,4′-dichlorobenzhydryloxy)piperidine (87) (0.27 mmol) in dichloromethane (10 mL) was added di-tert-butyl dicarbonate (0.30 mmol) and MP-carbonate (0.90 mmol) and the reaction mixture was stirred at ambient temperature for 17 hours. Elution through a pre-wetted (dichloromethane) SCX-2 (2 g) cartridge with dichloromethane and evaporation afforded the title compound as a colourless oil (28 mg, 21%).

MS 436 [M+H]⁺

LC (50/80) 95.9%, 8.41 min

### Example 63

### 4-(2,4′-dichlorobenzhydryloxy)-O-(benzyl)piperidine-1-carboxylate (109)

This material was prepared from 4-(2,4′-dichlorobenzhydryloxy)piperidine (87) and benzyl chloroformate using the method described for 4-(2,4′-dichlorobenzhydryloxy)-N-(tert-butyl)piperidine-1-carbamate (108) (19 mg, 10%)

NMR (400 MHz, CDCl₃) δ_{H} 1.60 (2H, br s), 1.81 (2H, br s), 3.14 (2H, m), 3.58 (1H, m), 3.79 (2H, br m), 5.15 (2H, s), 5.49 (1H, s), 7.21-7.40 (12H, m)

LC (50/80) 91.7%, 9.01 min

### Example 64

### 4-(4,4′-dichlorobenzhydryloxy)-O-(tert-butyl)piperidine-1-carboxylate (110)

This material was prepared from 4-(4,4′-dichlorobenzhydryloxy)piperidine (104) and di-tert-butyl dicarbonate using the method described for 4-(2,4′-dichlorobenzhydryloxy)-N-(tert-butyl)piperidine-1-carbamate (108) (144 mg, 79%)

NMR (400 MHz, CDCl₃) δ_{H} 1.45 (9H, s), 1.60 (2H, br m), 1.79 (2H, br m), 3.11 (2H, m), 3.53 (1H, m), 3.67 (2H, br s), 5.46 (1H, s), 7.16-7.31 (8H, m)

LC (50/80) 98.3%, 6.95 min

### Preparation of 2,2′-dichlorobenzhydrol (111)

Sodium borohydride (22.2 mmol) was added portionwise to a stirred solution of 2,2′-dichlorobenzophenone (20.2 mmol) in ethanol (100 ml) at 0° C. Stirring was continued for 30 mins then the reaction mixture was allowed to warm to ambient temperature. After a further 1 hr, the excess borohydride was quenched by the addition of saturated ammonium chloride. The organic phase was separated, dried (MgSO₄) and evaporated, under reduced pressure, to afford the title compound as an oil which solidified on standing (5.02 g, 98%).

### Preparation of 1-benzyl-4-(2,2′-dichlorobenzhydryloxy)piperidine (112)

To a solution of N-benzyl-4-piperidinol (9.92 mmol) in toluene (30 ml) was added 2,2′-dichlorobenzhydrol (111) (19.8 mmol) and para-toluenesulfonic acid (19.8 mmol). The reaction mixture was heated to reflux for 19 brs, cooled and partitioned between ethyl acetate and aq. sodium bicarbonate. The organic phase was separated and washed with water and brine, dried (MgSO₄) and concentrated under reduced pressure to afford the crude product, which was diluted with iso-hexane. After stirring for 1 hr, the white solid that had precipitated was removed by filtration (bis-ether by-product) and the liquors concentrated under reduced pressure to afford the title compound as a pale yellow oil (4.79 g).

MS 426 [M+H]⁺

LC (50/80) 86.3%, 7.73 min

### Preparation of 4-(2,2′-dichlorobenzhydryloxy)piperidine (113)

This material was prepared from 1-benzyl-4-(2,2′-dichlorobenzhydryloxy)piperidine (112) (4.76 mmol) using the method described for the preparation of 4-(2,4′-dichlorobenzhydryloxy)piperidine (87). Trituration of the initial product with diethyl ether afforded the title compound as a white solid (1.36 g, 77%).

NMR (400 MHz, d₆ DMSO) δ_{H} 1.80 (2H, m), 2.05 (2H, m), 2.90 (2H, m), 3.18 (2H, m), 3.75 (1H, m), 6.20 (1H, s), 7.38-7.45 (8H, m), 8.75-8.80 (2H, br).

LC (50/80) 98.1%, 2.84 min

### Example 65

### 4-(2,2′-dichlorobenzhydryloxy)-N-(3-chloro-4-methoxyphenyl)piperidine-1-carboxamide (114)

To a stirred solution of 4-(2,2′-dichlorobenzhydryloxy)piperidine (113) (0.27 mmol) in dichloromethane (10 mL) was added 3-chloro-4-methoxyphenyl isocyanate (0.27 mmol) and triethylamine (0.40 mmol) and the reaction mixture was stirred at ambient temperature for 17 hours. The reaction mixture was eluted through a pre-wetted (dichloromethane) SCX-2 (2 g) cartridge with dichloromethane/methanol (1:1) and evaporated to yield the title compound as a colourless gum (29 mg, 21%).

NMR (400 MHz, CDCl₃) δ_{H} 1.91 (2H, br m), 2.08 (2H, br m), 3.28 (2H, br m), 3.79 (3H, br m), 3.87 (3H, s), 5.26 (2H, s), 6.31 (1H, s), 6.85 (1H, d), 7.25-7.52 (11H, m)

LC (50/80) 98.2%, 7.70 min

### Example 66

### 4-(2,2′-dichlorobenzhydryloxy)-N-(3-chlorophenyl)piperidine-1-carboxamide (115)

To a stirred solution of 4-(2,2′-dichlorobenzhydryloxy)piperidine (113) (0.27 mmol) in dichloromethane (10 mL) was added 3-chlorophenyl isocyanate (0.27 mmol) and triethylamine (0.40 mmol) and the reaction mixture was stirred at ambient temperature for 17 hours. The reaction mixture was eluted through a pre-wetted (dichloromethane) SCX-2 (2 g) cartridge with dichloromethane/methanol (1:1) and evaporated to yield the title compound as a pale pink solid (40 mg, 31%).

NMR (400 MHz, CDCl₃) δ 1.82 (2H, m), 1.93 (2H, m), 3.26 (2H, m), 3.78 (3H, m), 6.35 (1H, s), 6.40 (1H, s), 7.22-7.49 (12H, m)

LC (50/80) 82.7%, 8.19 min

### Preparation of 1-benzyl-(3R)-{(R/S)-(4-chlorophenyl)-[2-(trifluoromethyl)phenyl]methoxy}pyrrolidine (116)

This material was prepared from (R)-(+)-1-benzyl-3-pyrrolidinol and 4-chloro-2′-(trifluoromethyl)benzhydrol (13), using the procedure described for (R)-1-benzyl-3-(4,4′-dichlorobenzhydryloxy)pyrrolidine (73) (9.84 g, 83%).

MS 446 [M+H]⁺

LC (50/80) 97.5%, 7.97 min

### Preparation of (3R)-{(R/S)-(4-chlorophenyl)-[2-(trifluoromethyl)phenyl]methoxy}pyrrolidine (117)

This material was prepared from 1-benzyl-(3R)-{(R/S-(4-chlorophenyl)-[2-(trifluoromethyl)phenyl]methoxy}pyrrolidine (115), using the procedure described for (R)-3-(4,4′-dichlorobenzhydryloxy)pyrrolidine (74) (3.52 g, 46%).

MS 356 [M+H]⁺

LC (50/80) 98.8%, 3.83 min

### Example 67

### (3R)-{(R/S)-(4-chlorophenyl)-[2-(trifluoromethyl)phenyl]methoxy}-N-(tert-butyl)pyrrolidine-1-carboxamide (118)

This material was prepared from (3R)-{(R/S)-(4-chlorophenyl)-[2-(trifluoromethyl)phenyl]methoxy}pyrrolidine (117) and tert-butyl isocyanate, using the procedure described for (R)-3-(4,4′-dichlorobenzhydryloxy)-N-(allyl)pyrrolidine-1-carboxamide (75). Purification by flash column chromatography [SiO₂; ethyl acetate-isohexane (20:80)→(30:70)] afforded the desired product (552 mg, 77%).

MS 455 [M+H]⁺

LC (50/80) 99.8%, 6.93 min

### Preparation of 1-benzyl-(3)-{(R/S)-(4-chlorophenyl)-[2-(trifluoromethyl)phenyl]methoxy}pyrrolidine (119)

This material was prepared from (S)-(−)-1-benzyl-3-pyrrolidinol and 4-chloro-2′-(trifluoromethyl)benzhydrol (13), using the procedure described for (R)-1-benzyl-3-(4,4′-dichlorobenzhydryloxy)pyrrolidine (73). The crude product was partially purified by flash column chromatography [SiO₂; isohexane→ethyl acetate-isohexane (1:4)] to remove residual (S′)-(−)-1-benzyl-3-pyrrolidinol and afforded an amber oil (13.55 g) which was taken on without further purification.

### Preparation of (3S)-{(R/S)-(4-chlorophenyl)-[2-(trifluoromethyl)phenyl]methoxy}pyrrolidine (120)

This material was prepared from crude 1-benzyl-(35)-{(R/S)-(4-chlorophenyl)-[2-(trifluoromethyl)phenyl]methoxy}pyrrolidine (119), using the procedure described for (R)-3-(4,4′-dichlorobenzhydryloxy)pyrrolidine (74) (2.53 g, 37% over both steps).

MS 356 [M+H]⁺

LC (50/80) 97.9%, 3.88 min

### Example 68

### (3S)-{(R/S)-(4-chlorophenyl)-[2-(trifluoromethyl)phenyl]methoxy}-N-(tert-butyl)pyrrolidine-1-carboxamide (121)

This material was prepared from (3S)-{(R/S)-(4-chlorophenyl)-[2-(trifluoromethyl)phenyl]methoxy}pyrrolidine (120) and tert-butyl isocyanate, using the procedure described for (R)-3-(4,4′-dichlorobenzhydryloxy)-N-(allyl)pyrrolidine-1-carboxamide (75). Purification by flash column chromatography [SiO₂; ethyl acetate-isohexane (20:80)→(25:75)] afforded the desired product (523 mg, 80%).

MS 455 [M+H]⁺

LC (50/80) 99.8%, 6.93 min

### Example 69

### (3S)-{(R*)-(4-chlorophenyl)-[2-(trifluoromethyl)phenyl]methoxy}-N-(tert-butyl)pyrrolidine-1-carboxamide (122) and

### Example 70

### (3S)-[(R*)-(4-chlorophenyl)-[2-(trifluoromethyl)phenyl]methoxy]-N-(tert-butyl)pyrrolidine-1-carboxamide (123)

The diastereomeric compound (3S)-{(R/S)-(4-chlorophenyl)-[2-(trifluoromethyl)phenyl]methoxy}-N-(tert-butyl)pyrrolidine-1-carboxamide (121) was separated into samples which were significantly enriched in each single diastereomeric form. Thus, a sample of the diastereomeric mixture (500 mg) was separated by repeat injection onto a Daicel Chiralpak® AD™ chiral HPLC column (250 mm×21 nm ID) fitted with a guard column (50 mm×21 mm ID) [eluent: IPA—hexane (10:90); flow-rate: 10 mL/min; wavelength: 235 nm]. The combined first-eluting diastereomer [arbritrarily assigned as the (S,R*) diastereomer] fractions were evaporated to afford (3S)-{(R*)-(4-chlorophenyl)-[2-(trifluoromethyl)phenyl]methoxy}-N-(tert-butyl)pyrrolidine-1-carboxamide (122) as an off-white foam (182.5 mg, 36.5%).

MS 455 [M+H]⁺

LC (50/80) 99.8%, 6.95 min

LC [Chiral AD; IPA—hexane (10:90)] 98.3%, 6.71 min; 100% de

The combined second-eluting diastereomer [arbritrarily assigned as the (S,S*) diastereomer] fractions were evaporated to afford (3S)-{(S*)-(4-chlorophenyl)-[2-(trifluoromethyl)phenyl]methoxy}-N-(tert-butyl)pyrrolidine-1-carboxamide (123) as an off-white foam (198.2 mg, 39.6%).

MS 455 [M+H]⁺

LC (50/80) 99.8%, 6.93 min

LC [Chiral AD; IPA—hexane (10:90)] 95.6%, 7.87 min; 3.1%, 6.76 min; 93.8% de

### Preparation of 1-(1-(4-chlorophenyl)-1-hydroxy)methyl-cyclohexane (124)

This material was prepared from 4-chlorobenzaldehyde and cyclohexylmagnesium chloride, using the procedure described for 2,4,4′-trichlorobenzhydrol (8) (7.05 g, 90%).

NMR (400 MHz, CDCl₃) δ_{H} 0.98-0.83 (1H, m), 1.10-1.00 (1H, m), 1.28-1.12 (3H, m), 1.44-1.33 (1H, m), 1.72-1.50 (3H, m), 1.78-1.74 (1H, m), 1.81 (1H, d, J=3.4 Hz), 1.99-1.86 (1H, m), 4.36 (1H, dd, J 7.0, 3.0 Hz), 7.23 (2H, d, J=8.5 Hz), 7.30 (2H, d, J=8.5 Hz). IR□max; diffuse reflectance KBr/cm⁻¹; 3384, 2927, 2853, 1598, 1492, 1450, 1410, 1090, 1014, 893, 833, 550.

### Preparation of 1-benzhydryl-3-(1-(4-chlorophenyl)-1-cyclohexyl)methoxy-azetidine (125)

This material was prepared from 1-(1-(4-chlorophenyl)-1-hydroxy)methyl-cyclohexane (124) and 1-benzhydryl-3-azetidinol (1) using the procedure described for compound (3); (1.66 g, 47%).

MS 446/448 [M+H]⁺

LC (50/80) 86%, 3.87 min

### Preparation of 3-(1-(4-chlorophenyl)-1-cyclohexyl)methoxy-azetidine (126)

This material was prepared from 1-benzhydryl-3-(1-(4-chlorophenyl)-1-cyclohexyl)methoxy-azetidine (125), using the procedure as described for 3-(2,4′-dichlorobenzhydryloxy)azetidine hydrochloride (4); (1.30 g) and the material was taken on without full purification.

### Example 71

### Preparation of 3-(1-(4-chlorophenyl)-1-cyclohexyl)methoxy-(1-piperidine-carbonyl) azetidine (127)

This material was prepared from 3-(1-(4-chlorophenyl)-1-cyclohexyl)methoxy-azetidine (126) and piperidine, using the procedure as described for compound (16); (0.121 g, 58%)

MS 405/407 [M+NH₄]⁺

LC (50/80) 99.7, 7.71 min

1. A method of treatment of disorders comprising administration to a subject in need of such treatment an effective dose of a compound of formula (1) or a pharmaceutically acceptable salt or prodrug thereof for the treatment of disorders mediated by CB1 receptors: 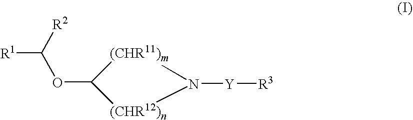
wherein:
R¹ is aryl or heteroaryl;
R² is alkyl, aryl or heteroaryl;
R³ is alkyl, aryl, heteroaryl, NR⁹R¹⁰, OR¹⁵, or NR¹⁶C(O)R¹⁷;
Y is C═O, C═S, SO₂, or (CR⁷R⁸)ₚ;
R⁷ and R⁸ are independently selected from H and lower alkyl;
R⁹ is selected from alkyl, aryl, heteroaryl, and non-aromatic heterocyclic groups, or together with R¹¹ forms a saturated 4, 5, 6 or 7 membered ring optionally containing an additional heteroatom selected from N and O;
R¹⁰ is selected from H and lower alkyl, or together with R⁹ forms a saturated 4, 5, 6 or 7 membered ring optionally containing an additional heteroatom selected:from N and O;
R¹¹ and R¹² are independently selected from H and lower alkyl;
R¹⁵ is selected from alkyl and aryl;
R¹⁶ is selected from H and lower alkyl;
R¹⁷ is selected from alkyl, aryl and heteroaryl;
m=1 or 2;
n=1 or 2; and
p=1, 2, 3 or 4,
wherein if —Y—R³ is —C(O)NH(alkyl) then:
R¹ and/or R² is selected from heteroaryl; and/or
m and/or n is 2; and/or
R¹¹ and/or R¹² is lower alkyl.

2. A method according to claim 1 wherein the disorder is selected from psychosis, memory deficit, cognitive disorders, attention deficit disorder, migraine, neuropathy, neuro-inflammatory disorders, cerebral vascular injuries, head trauma, anxiety disorders, depression, stress, epilepsy, dementia, disfonia, Alzheimer's disease, Huntingdon's disease, Tourette's syndrome, pain, Parkinson's disease, schizophrenia, substance abuse disorders, smoking cessation, treatment of nicotine dependance and/or treatment of symptoms of nicotine withdrawal, gastrointestinal disorders, eating disorders associated with excessive food intake, and non-insulin dependant diabetes mellitus.

3. A method according to claim 2 wherein said substance abuse disorder is abuse of nicotine, alcohol and/or opiates.

4. A method according to claim 2 wherein said eating disorder is obesity.

5. A method according to claim 2 wherein said disorder is Parkinson's Disease.

6. A method according to claim 2 for smoking cessation.

7. A method according to claim 2 for gastrointestinal disorders.

8. A method according to claim 2 wherein said disorder is selected from psychosis, schizophrenia, cognitive disorders, attention deficit disorder, smoking cessation, gastrointestinal disorders, eating disorders associated with excessive food intake, and non-insulin dependant diabetes mellitus.

9. (canceled)

10. A method according to claim 1 wherein in the compound of formula (I) R¹ and R² are independently selected from mono-cyclic aryl and heteroaryl groups.

11. A method according to claim 1 wherein in the compound of formula (I) R¹ and R² are independently selected from a group A(R⁴)(R⁵)(R⁶), where A is an aryl or heteroaryl ring, and R⁴, R⁵ and R⁶ are independently selected from hydrogen, halo, alkyl, thioalkyl, alkoxy, alkylsulfonyl, amino, mono- and di-alkyl amino, mono- and di-aryl amino, alkylarylamino, aminoalkyl, alkylaminoalkyl, dialkylaminoalkyl, NR¹⁸C(O)R¹⁹, NR¹⁸SO₂R²⁰, COOR¹⁹, OC(O)R²⁰, CONR¹³R¹⁴ and SO₂NR¹³R¹⁴, wherein R¹³ and R¹⁴ are independently selected from hydrogen and alkyl or may form a 5 or 6 membered ring optionally containing 1 or 2 additional heteroatoms selected from N, O and S; and wherein R¹⁸ is selected from H and lower alkyl, R¹⁹ is selected from H, alkyl, aryl and heteroaryl and R²⁰ is selected from alkyl, aryl and heteroaryl.

12. A method according to claim 11 wherein A is selected from phenyl, naphthyl, thienyl, furanyl, pyrrolyl; imidazolyl, pyrazolyl, thiazolyl, isothiazolyl, oxazolyl, isooxazolyl, pyridyl, pyrazinyl, pyrimidinyl, quinolinyl, isoquinolinyl, benzofuranyl and isobenzofuryl.

13. A method according to claim 1 wherein in the compound of formula (I) R¹ and R² are independently selected from phenyl.

14. A method according to a claim 1 wherein in the compound of formula (I) R¹ and R² is aryl and the other is heteroaryl, or both R¹ and R² are aryl.

15. A method according to a claim 1 wherein in the compound of formula (I) R¹ and R² are different.

16. A method according to claim 11 wherein in the compound of formula (I) R⁴, R⁵ and R⁶ are independently selected from fluoro, chloro, bromo and iodo.

17. A use or method according to claim 11 wherein in the compound of formula (I) R⁴, R⁵ and R⁶ are independently selected from alkyl, thioalkyl, alkoxy and alkylsulfonyl wherein the alkyl group is selected from lower alkyl.

18. A method according to claim 11 wherein in the compound of formula (I) R⁴, R⁵ and R⁶ are independently selected from trifluoromethyl and difluoromethoxy.

19. A method according to claim 11 wherein in the compound of formula (I) one or two of R⁴, R⁵ and R⁶ are hydrogen.

20. A method according to claim 11 wherein in the compound of formula (I) R¹⁸ is selected from H.

21. A method according to claim 11 wherein in the compound of formula (I) R¹⁹ and R²⁰ are independently selected from alkyl.

22. A method according to claim 11 wherein in the compound of formula (I) R¹⁹ and R²⁰ are independently selected from lower alkyl.

23. A method according to claim 11 wherein in the compound of formula (I) at least one of the R¹ and R² groups has a non-hydrogen substituent in the ortho-position(s) relative to the point of attachment to the [—CH—O—] group.

24. A method according to claim 1 wherein in the compound of formula (I) Y is C═O.

25. A method according to claim 1 wherein in the compound of formula (I) Y is selected from SO₂ and R³ is selected from alkyl, aryl and heteroaryl.

26. A method according to claim 1 wherein in the compound of formula (I) Y is selected from (CR⁷R⁸)p, p is 1, and R³ is selected from alkyl, aryl and heteroaryl.

27. A method according to claim 26 wherein in the compound of formula (I) R⁷ and/or R⁸ are hydrogen and p is 1.

28. A method according to claim 26 wherein in the compound of formula (I) R⁷ and/or R⁸ are hydrogen and p is 1.

29. A method according to claim 1 wherein in the compound of formula (I) R³ is selected from NR⁹R¹⁰.

30. A method according to claim 29 wherein in the compound of formula (I) R⁹ is a non-aromatic heterocyclic group selected from piperidinyl and morpholinyl.

31. A method according to claim 29 wherein in the compound of formula (I) either
(i) R⁹ is selected from aryl, heteroaryl and a non-aromatic heterocyclic group, and R¹⁰ is selected from H and lower alkyl; or
(ii) R⁹ is selected from alkyl and R¹⁰ is selected from lower alkyl; or
(iii) R⁹ and R¹⁰ form a 4, 5, 6 or 7-membered ring.

32. A method according to claim 1 wherein in the compound of formula (I) m is 1 and/or n is 1.

33. A method according to claim 1 wherein in the compound of formula (I) m and n are 1.

34. A method according to claim 1 wherein in the compound of formula (I) m is 2 and at least one of the R¹¹ groups in the (CHR¹¹)₂ moiety is hydrogen.

35. A method according to claim 1 wherein in the compound of formula (I) at least one of the R¹² groups in the (CHR²)₂ moiety is hydrogen.

36. A method according to claim 1 wherein in the compound of formula (I) R¹¹ and R¹² are independently selected from hydrogen.

37. A method according to claim 1 wherein in the compound of formula (I) R¹⁵ is selected from lower alkyl, phenyl and benzyl.

38. A method according to claim 1 wherein in the compound of formula (I) R¹⁶ is hydrogen.

39. A method according to claim 1 wherein in the compound of formula (I) R¹⁷ is lower alkyl or phenyl.

40. A method according to claim 1 wherein the compound is selected from:
4-[3-(2,4′-dichlorobenzhydryloxy)azetidine-1-carbonyl]-[1,4]diazepane-1-carboxylic acid tert-butyl ester
3-(2,4′-dichlorobenzhydryloxy)-N-methyl-N-pentyl-azetidine-1-carboxamide
3-(2,4,4′-trichlorobenzhydryloxy)-N-methyl-N-benzyl-azetidine-1-carboxamide
3-[2-(trifluoromethyl)-4′-chlorobenzhydryloxy]-N-methyl-N-benzyl-azetidine-1-carboxamide
3-(2-methyl-4′-chlorobenzhydryloxy)-N-methyl-N-benzyl-azetidine-1-carboxamide
3-(2,4′-dichlorobenzhydryloxy)-N-(tert-butyl)-azetidine-1-thiocarboxamide
3-[(S*)-2,4′-dichlorobenzhydryloxy]-N-(tert-butyl)-azetidine-1-thiocarboxamide
3-[2-(trifluoromethyl)-4′-chlorobenzhydryloxy]-N-(tert-butyl)-azetidine-1-thiocarboxamide
3-(2,4,4′-trichlorobenzhydryloxy)-1-[(S)-2-methoxy-2-phenylethanoyl]azetidine
3-(2,4′-dichlorobenzhydryloxy)-1-(3-chlorothiophen-2-yl-formyl)azetidine
1-[2-(tert-butyl)acetyl]-3-[2-(trifluoromethyl)-a-(2,3-dihydrobenzo[1,4]dioxin-6-yl)benzyloxy]azetidine
1-(4-nitrophenylsulfonyl)-3-[2-(trifluoromethyl)-4′-chlorobenzhydryloxy]azetidine
1-(thiophen-3-ylsulfonyl)-3-[2-(trifluoromethyl)-4′-chlorobenzhydryloxy]azetidine
1-(3-fluorophenylsulfonyl)-3-[2-(trifluoromethyl)-4′-chlorobenzhydryloxy]azetidine
1-phenylsulfonyl-3-(2,4,4′-trichlorobenzhydryloxy)azetidine
1-(n-butyl)sulfonyl-3-(2,4,4′-trichlorobenzhydryloxy)azetidine
3-[2-(trifluoromethyl)-4′-chlorobenzhydryloxy]-N-(4-chloro-3-nitrophenyl)azetidine-1-carboxamide
3-[2-(trifluoromethyl)-4′-chlorobenzhydryloxy]-N-(2,4-difluorophenyl)azetidine-1-carboxamide
3-[2-(trifluoromethyl)-4′-chlorobenzhydryloxy]-N-(2,3-dihydrobenzo[1,4]dioxin-6-yl)azetidine-1-carboxamide
3-(2,4′-dichlorobenzhydryloxy)-N-(2,6-difluorophenyl)azetidine-1-carboxamide
3-[4-chloro-a-(2-chloropyrid-3-yl)benzyloxy]-N-(1-adamantyl)azetidine-1-carboxamide
3-[2-chloro-a-(2-chloropyridin-5-yl)benzyloxy]-N-(tert-butyl)azetidine-1-carboxamide
3-[(S*)-4-chloro-a-(2-chloropyridin-3-yl)benzyloxy]-N-(tert-butyl)azetidine-1-carboxamide
3-[2-(trifluoromethyl)-4′-chlorobenzhydryloxy]-N-(benzoyl)azetidine-1-carboxamide
3-(2-(trifluoromethyl)-4′-fluorobenzhydryloxy]-N-(benzoyl)azetidine-1-carboxamide
3-(2,4′-dichlorobenzhydryloxy)-N-(piperidyl)azetidine-1-carboxamide
4-(2,4′-dichlorobenzhydryloxy)-N-(1-adamantyl)piperidine-1-carboxamide
1-(1-piperidinecarbonyl)-4-(2,4′-dichlorobenzhydryloxy)piperidine
4-(2,4,4′-trichlorobenzhydryloxy)-N-(tert-butyl)piperidine-1-carboxamide
4-(2,4,4′-trichlorobenzhydryloxy)-N-(cyclohexyl)piperidine-1-carboxamide
4-(2,4,4′-trichlorobenzhydryloxy)-N-methyl-N-benzylpiperidine-1-carboxamide
1-(1-piperidinecarbonyl)-4-(2,4,4′-trichlorobenzhydryloxy)piperidine, and
1-(tert-butylacetyl)-4-(2,4′-dichlorobenzhydryloxy)piperidine.

41. A compound of formula (I): 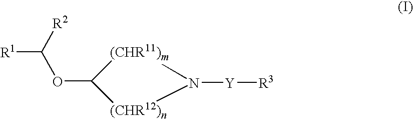
wherein:
R¹ is aryl or heteroaryl;
R² is alkyl, aryl or heteroaryl;
R³ is alkyl, aryl, heteroaryl, NR⁹R¹⁰, OR¹⁵, or NR¹⁶C(O)R¹⁷;
Y is C═O, C═S, or SO₂;
R⁹ is selected from alkyl, aryl, heteroaryl, and non-aromatic heterocyclic groups, or together with R¹⁰ forms a saturated 4, 5, 6 or 7 membered ring optionally containing an additional heteroatom selected from N and O;
R¹⁰ is selected from H and lower alkyl, or together with R⁹ forms a saturated 4, 5, 6 or 7 membered ring optionally containing an additional heteroatom selected from N and O;
R¹¹ and R¹² are independently selected from H and lower alkyl;
R¹⁵ is selected from alkyl and aryl;
R¹⁶ is selected from H and lower alkyl;
R¹⁷ is selected from alkyl, aryl and heteroaryl;
m=1 or 2;
n=1 or 2; and
p=1, 2, 3 or 4,
or a pharmaceutically acceptable salt or prodrug thereof, wherein if —Y—R³ is —C(O)NH(alkyl) then:
R¹ and/or R² is selected from heteroaryl; and/or
m and/or n is 2;
and/or R¹¹ and/or R¹² is lower alkyl.

42-69. (canceled)
